(19) United States
(12) Patent Application Publication
(10) Pub. No.: US 2024/0101971 A1
(43) Pub. Date: Mar. 28, 2024

(54) MICROTUBULE DESTABILIZER ADDITIVES TO INCREASE RECOMBINANT VIRAL VECTOR TITERS

(71) Applicant: Biogen MA Inc., Cambridge, MA (US)

(72) Inventors: Terrence Michael Dobrowsky, Waltham, MA (US); Jenny Wang-Yuanzhen Shupe, Littleton, MA (US)

(21) Appl. No.: 18/273,053

(22) PCT Filed: Jan. 21, 2022
(86) PCT No.: PCT/US2022/013310

§ 371 Date: Jul. 19, 2023

Related U.S. Application Data
(60) Provisional application No. 63/140,705, filed on Jan. 22, 2021.

(51) Int. Cl. C12N 7/00; C12M 1/00; C12N 15/85

(52) U.S. Cl. CPC C12N 7/00; C12M 29/10; C12N 15/85; C12N 2750/14122; C12N 2750/14123; C12N 2750/14152; C12N 2800/107

(57) ABSTRACT

The present disclosure pertains to methods and compositions for the production of recombinant viral vectors, e.g., recombinant adeno-associated viruses (rAAVs), using at least one microtubule destabilizing agent described herein.

### CROSS REFERENCE TO RELATED APPLICATIONS

This application claims priority to U.S. Provisional Patent Application No. 63/140,705, filed on Jan. 22, 2021, which is incorporated herein by reference in its entirety.

### BACKGROUND

Recombinant viral vector mediated gene therapy is a rapidly developing therapeutic field. For example, recombinant adeno-associated viruses (rAAVs) are engineered by deleting, in whole or in part, an internal portion of an AAV genome and inserting a heterologous nucleic acid of interest between inverted terminal repeats (ITRs) in a vector. ITRs remain functional in such vectors, thereby allowing for replication and packaging of rAAV particles encoding a payload enclosed within an AAV capsid. A major challenge in development of recombinant viral vectors (e.g., rAAVs) as gene therapies is manufacturing technologies that yield the quantity and quality of recombinant viral vector particles needed for clinical applications.

Thus, there remains a need for improved methods and compositions for manufacturing recombinant viral vectors (e.g., rAAVs). In particular, there is a need for methods and compositions that can be used to efficiently produce high viral titers of recombinant viral vectors (e.g., rAAVs) for gene therapy products.

### SUMMARY

The present disclosure encompasses, inter alia, methods, compositions, and systems for producing a plurality of recombinant viral vector particles (e.g., recombinant adeno-associated virus (rAAV) particles) that use at least one microtubule destabilizing agent. Microtubules are polymers of tubulin and a major component of mitotic spindles, which separate duplicated chromosomes during cell division. If microtubules are destabilized, such as by the use of at least one microtubule destabilizing agent, a cell cannot divide into daughter cells. In some embodiments, a microtubule destabilizing agent described herein arrests cell cycle of host cells (e.g., HEK293 cells, CHO-K cells, HeLa cells, or a variant thereof). In some embodiments, a microtubule destabilizing agent described herein comprises or is a G2/M inhibitor (e.g., colcemid, colchicine, vinblastine, vincristine, or a combination, derivative, or salt thereof). In some embodiments, a microtubule destabilizing agent described herein comprises or is a Vinca alkaloid (e.g., vinblastine or vincristine, or a combination, derivative, or salt thereof).

A plurality of recombinant viral vector particles (e.g., rAAV particles) produced using at least one microtubule destabilizing agent described herein can have one or more improved characteristics. Such improved characteristics can include, but are not limited to, higher titer (e.g., genome titer) of recombinant viral vector particles (e.g., rAAV particles) produced, e.g., relative to recombinant viral vector particles (e.g., rAAV particles) produced under the same conditions and in the same medium, but without at least one microtubule destabilizing agent described herein. Such improved characteristics can include, but are not limited to, improved product qualities, such as improved product potency and empty/full ratio and thus benefits product purification process and increase overall yield, e.g., relative to recombinant viral vector particles (e.g., rAAV particles) produced under the same conditions and in the same medium, but without at least one microtubule destabilizing agent described herein.

In one aspect, the disclosure provides methods of producing a plurality of recombinant adeno-associated virus (rAAV) particles comprising: (a) transfecting host cells with one or more vectors encoding: (i) at least one payload flanked by an inverted terminal repeat (ITR) on either side of the at least one payload, (ii) at least one Rep polypeptide, (iii) at least one Cap polypeptide, and (vii) at least one helper polypeptide, in medium; (b) adding at least one microtubule destabilizing agent to the medium; and (c) culturing the host cells under conditions suitable for production of the plurality of AAV particles, thereby producing the plurality of rAAV particles.

In some embodiments, a microtubule destabilizing agent comprises or is a G2/M inhibitor. In some embodiments, a microtubule destabilizing agent comprises or is colcemid, colchicine, vinblastine, vincristine, or a combination, derivative, or salt thereof.

In some embodiments, a microtubule destabilizing agent is added to medium prior to transfection of host cells with one or more vectors. In some embodiments, a microtubule destabilizing agent is added to medium substantially simultaneously with one or more vectors. In some embodiments, a microtubule destabilizing agent is added to medium after transfection of host cells with one or more vectors. In some embodiments, a microtubule destabilizing agent is added to medium at about 24 hours to about 1 minute prior to transfection. In some embodiments, a microtubule destabilizing agent is added to medium at about 24 hours to about 1 minute after transfection.

In some embodiments, a plurality of rAAV particles are produced at a higher titer. In some embodiments, a higher titer is relative to rAAV particles produced under the same conditions and in the same medium, but without a microtubule destabilizing agent. In some embodiments, a higher titer comprises or is about a 1-fold higher titer to about a 6-fold higher titer.

In some embodiments, one or more vectors comprise: (i) a first vector encoding at least one payload flanked by an ITR on either side of the at least one payload, (ii) a second vector encoding at least one Rep polypeptide and at least one Cap polypeptide, and (iii) a third vector encoding at least one helper polypeptide.

In some embodiments, one or more vectors comprise: (i) a first vector encoding at least one Cap polypeptide and at least one payload flanked by an ITR on either side of the at least one payload; and (ii) a second vector encoding at least one helper polypeptide and at least one Rep polypeptide.

In some embodiments, at least one helper polypeptide comprises one, two, three, or four of E1, E2A, E4orf6, or VA RNA polypeptides.

In some embodiments, host cells comprise or are adherent cells. In some embodiments, host cells comprise or are suspension cells. In some embodiments, host cells comprise or are mammalian cells. In some embodiments, mammalian cells comprise or are HEK293 cells, CHO-K cells, HeLa cells, or a variant thereof. In some embodiments, host cells comprise or express an E1 polypeptide.

In some embodiments, one or more vectors are transfected into host cells in the presence of polyethylenimine (PEI).

In another aspect, the disclosure provides reaction mixtures comprising one or more vectors encoding: (i) at least one payload flanked by an inverted terminal repeat (ITR) on either side of the at least one payload, (ii) at least one Rep polypeptide, (iii) at least one Cap polypeptide, and (vii) at least one helper polypeptide; and a microtubule destabilizing agent.

In some embodiments, one or more vectors comprises: (i) a first vector encoding at least one payload flanked by an ITR on either side of the at least one payload, (ii) a second vector encoding one at least one Rep polypeptide and at least one Cap polypeptide, and (iii) a third vector encoding at least one helper polypeptide. In some embodiments, one or more vectors comprises: (i) a first vector encoding at least one Cap polypeptide and at least one payload flanked by an ITR on either side of the at least one payload; and (ii) a second vector encoding at least one helper polypeptide and at least one Rep polypeptide.

In another aspect, the disclosure provides cultures comprising a plurality of host cells and a reaction mixture of any aspect or embodiment described herein.

In another aspect, the disclosure provides bioreactors comprising a culture of any aspect or embodiment described herein. In some embodiments, a bioreactor comprises or is a continuous flow bioreactor, a batch process bioreactor, a perfusion bioreactor, or a fed batch bioreactor.

Other features, objects, and advantages of the present disclosure are apparent in the detailed description that follows. It should be understood, however, that the detailed description, while indicating embodiments of the present disclosure, is given by way of illustration only, not limitation. Various changes and modifications within the scope of the disclosure will become apparent to those skilled in the art from the detailed description.

### BRIEF DESCRIPTION OF THE DRAWINGS

The Figures described below, which together make up the Drawings, are for illustration purposes only, not for limitation.

FIG. 1 is a graph showing fold changes in titer (e.g., genome titer) of recombinant adeno-associated virus (rAAV) particles following transient transfection of suspension HEK293 cells with plasmids encoding Payload C flanked by an inverted terminal repeat (ITR) on either side, at least one Rep polypeptide, at least one Cap polypeptide, and at least one helper polypeptide as well as addition of cell cycle arresting agents (aphidicolin, caffeine, colcemid, colchicine, flavopiridol, hydroxyurea, thymidine, trichostatin A, and vinblastine sulfate). Cell cycle arresting agents were added at 24 hours prior to transfection (24 h pre tfx) or at 0.5 hours after transfection (at tfx).

FIGS. 2A-2B are graphs showing fluorescence activated cell sorting (FACS) analysis of cell cycle phase of suspension HEK293 cell cultures (not transfected) after addition of cell cycle arresting agents (aphidicolin, caffeine, colcemid, colchicine, flavopiridol, hydroxyurea, thymidine, trichostatin A, and vinblastine sulfate) for 24 hours (FIG. 2A) and cell cycle arrest of suspension HEK293 cell cultures by colcemid in G2/M phase (FIG. 2B).

FIGS. 3A-3B are graphs showing fold changes in titer of rAAV particles following transient transfection of suspension HEK293 cells with plasmids encoding Payload C flanked by an ITR on either side, at least one Rep polypeptide, at least one Cap polypeptide, and at least one helper polypeptide as well as the addition of microtubule destabilizing agents (colcemid, colchicine, vinblastine sulfate, and vincristine sulfate) at 1× and 2× concentrations (FIG. 3A) and 2× and 5× concentrations (FIG. 3B) as shown in Table 1 of Example 1 herein, at 0.5 hours post-transfection.

FIG. 4 is a graph showing fold changes in titer of rAAV particles when microtubule destabilizing agents (colcemid, colchicine, and vinblastine sulfate) were added at different time points (3 hours, 2 hours, and 1 hour pre-transfection and 0.5 hour, 2 hour, and 3 hours post-transfection) around transient transfection of suspension HEK293 cells with plasmids encoding Payload C flanked by an ITR on either side, at least one Rep polypeptide, at least one Cap polypeptide, and at least one helper polypeptide.

FIG. 5 is a graph showing fold changes in titer of rAAV particles following transient transfection of HEK293 cells with plasmids encoding Payload C flanked by an ITR on either side, at least one Rep polypeptide, at least one Cap polypeptide, and at least one helper polypeptide, addition of microtubule destabilizing agents colcemid (0.4 μM concentration) or colchicine (5 μM concentration) at 0.5 hours post-transfection, and culturing in a 3-liter bioreactor for seven days.

FIG. 6 is a graph showing fold changes in titer of rAAV particles following transient transfection of adherent HEK293 cells with plasmids encoding Payload C flanked by an ITR on either side, at least one Rep polypeptide, at least one Cap polypeptide, and at least one helper polypeptide and addition of microtubule destabilizing agents (colcemid, colchicine, vinblastine sulfate, and vincristine sulfate) at 3 hours post-transfection and at different concentrations shown in Table 1 of Example 1 herein.

FIG. 7 is a graph showing fold changes in titer of rAAV particles following transient transfection of suspension HEK293 cells with plasmids encoding different payloads (Payloads A-C) flanked by an ITR on either side, at least one Rep polypeptide, at least one Cap polypeptide, and at least one helper polypeptide as well as addition of colcemid at 0.5 hours post-transfection. Dose response curves of about 0.005 μM to about 4 μM colcemid were generated for the plasmids encoding Payload A and Payload B. A concentration of about 0.4 μM colcemid was used for the plasmid encoding Payload C.

FIGS. 8A-8C are graphs showing fold changes in titer of rAAV particles following transient transfection of HEK293 cells with plasmids encoding Payload A flanked by an ITR on either side, at least one Rep polypeptide, at least one Cap polypeptide, and at least one helper polypeptide, addition of colcemid at 0.5 hours post-transfection, and culturing in batch bioreactors (FIG. 8A) or fed batch bioreactor (FIG. 8B-C) for manufacturing scale.

### DEFINITIONS

In order for the present disclosure to be more readily understood, certain terms are first defined below. Additional definitions for the following terms and other terms are set forth throughout the specification. The publications and other reference materials referenced herein to describe the background of the disclosure and to provide additional detail regarding its practice are hereby incorporated by reference.

In this application, unless otherwise clear from context, (i) the term “a” may be understood to mean “at least one”; (ii) the term “or” may be understood to mean “and/or”; and (iii) where ranges are provided, endpoints are included.

About or approximately: As used herein, the terms “about” or “approximately” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Adeno-associated virus (AAT): As used herein, the terms “Adeno-associated virus” and “AAV” refer to viral particles, in whole or in part, of family Parvoviridae and genus Dependoparvovirus. AAV is a small replication-defective, nonenveloped virus. AAV may include, but is not limited to, AAV serotype 1, AAV serotype 2, AAV serotype 3 (including serotypes 3A and 3B), AAV serotype 4, AAV serotype 5, AAV serotype 6, AAV serotype 7, AAV serotype 8, AAV serotype 9, AAV serotype 10, AAV serotype 11, AAV serotype 12, AAV serotype 13, AAV serotype rh10, AAV serotype rh74, AAV from the HSC 1-17 series, AAV from the CBr, CLv or CLg series, snake AAV, avian AAV, bovine AAV, canine AAV, equine AAV, ovine AAV, goat AAV, shrimp AAV, and any variant of any of the foregoing. AAV may also include engineered or chimeric versions of a wild-type AAV that include one or more insertions, deletions and/or substitutions within the Cap polypeptide(s) that affect one or more properties of the wild-type AAV serotype, including without limitation tropism and evasion of neutralizing antibodies (e.g., AAV-DJ, AAV-PHP.B, AAV-PHP.N, AAV.CAP-B1 to AAV.CAP-B25 and variants thereof). Wild-type AAV is replication deficient and requires co-infection of cells by a helper virus (e.g., adenovirus, herpes, or vaccinia virus) or supplementation of helper viral genes in order to replicate.

Agent: The term “agent,” as used herein, refers to a compound or entity of any chemical class including, for example, a small molecule, polypeptide, nucleic acid, saccharide, lipid, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them.

Bioreactor: The term “bioreactor,” as used herein, refers to any vessel used for the growth of a cell culture (e.g., a mammalian cell culture). A bioreactor can be of any size and/or any shape so long as it is useful for culturing a cell culture (e.g., a mammalian cell culture).

Cap polypeptide: As used herein, the term “Cap polypeptide” refers to structural proteins that form a functional AAV capsid, which can in turn package DNA and infect a target cell. In some embodiments, capsid polypeptides comprise VP1, VP2 and/or VP3. In some embodiments, Cap polypeptides comprise all AAV capsid subunits (e.g., VP1, VP2 and VP3), but less than all capsid subunits may be present as long as a functional capsid is produced. In some embodiments, a nucleic acid sequence encoding one or more Cap polypeptides is present on a single vector (e.g., plasmid). In some embodiments, a Cap polypeptide comprises an AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25 Cap polypeptide, or a variant of any of the foregoing. AAV capsid genes and proteins have been described in, e.g., Knipe et al., FIELDS VIROLOGY, Volume 1, (6th ed., Lippincott-Raven Publishers), which is hereby incorporated by reference in its entirety.

Cell Density: As used herein, the term “cell density” refers to that number of cells present in a given volume of medium or that number of cells present in a given surface area. For example, cell density may be represented as viable cells (vc)/ml or vc/cm² of culture medium.

Comprising: A composition or method described herein as “comprising” or “including” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Culture: As used herein, the terms “culture” and “cell culture” refer to a cell population (e.g., a eukaryotic cell population) that is suspended in or covered by a medium under conditions suitable to survival and/or growth of a cell population. As will be clear to those of ordinary skill in the art, these terms can also refer to a combination comprising a cell population and medium.

Derivative: As used herein, the term “derivative” refers to a structural analogue of a reference substance. That is, a “derivative” is a substance that shows significant structural similarity with the reference substance, such as sharing a core or consensus structure, but also differs in certain discrete ways. In some embodiments, a derivative is a substance that can be generated from a reference substance by chemical manipulation. In some embodiments, a derivative is a substance that can be generated through performance of a synthetic process substantially similar to (e.g., sharing a plurality of steps with) one that generates the reference sub stance.

Fragment: As used herein, the term “fragment” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Gene: As used herein, the term “gene” refers to a DNA sequence that codes for a product (e.g., an RNA product and/or a polypeptide product). In some embodiments, a gene includes a coding sequence that encodes a particular product. In some embodiments, a gene includes a non-coding sequence. In some embodiments, a gene includes both coding (e.g., exonic) and non-coding (e.g., intronic) sequence. In some embodiments, a gene includes one or more regulatory elements, e.g., that control or effect one or more aspects of gene expression (e.g., inducible gene expression).

Helper polypeptide: As used herein, the term “helper polypeptide” refers to a polypeptide necessary to generate functional rAAV particles derived from a helper adenovirus (e.g., an Ad5 helper virus, e.g., wild-type or recombinantly engineered Ad5 helper virus). Helper polypeptides can include E1A polypeptide, E1B polypeptide, E2A polypeptide, E4orf6 polypeptide, VA RNA polypeptide, and any variant or fragment of any of the foregoing. In certain embodiments, a vector encoding at least one helper polypeptide is transfected into an E1 complementing cell line (e.g., HEK293). A nucleotide sequence of an Ad5 helper polypeptide can be derived from the Adenovirus 5 genome (GenBank Accession No. AY601635).

Host cell: As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Host cells can include prokaryotic and eukaryotic cells selected from any Kingdom of life that are suitable for expressing an exogenous nucleic acid sequence (e.g., a recombinant nucleic acid sequence).

Exemplary host cells include, but are not limited to, prokaryotes and eukaryotes (e.g., single-cell or multiple-cell), bacterial cells (e.g., strains of E. coli, Bacillus spp., or Streptomyces spp.), mycobacteria cells, fungal cells, yeast cells (e.g., S. cerevisiae, S. pombe, P. pastoris, or P. methanolica), plant cells, insect cells (e.g., SF-9, SF-21, baculovirus-infected insect cells, or Trichoplusia ni), non-human animal cells, human cells, and cell fusions (e.g., hybridomas or quadromas). In some embodiments, a host cell comprises or is a human, monkey, ape, hamster, rat, or mouse cell. In some embodiments, a host cell is a eukaryotic cell chosen from: a CHO (e.g., CHO-K, CHO-DXB11, Veggie-CHO), COS (e.g., COS-7), retinal, Vero, CV1, kidney (e.g., HEK293 (e.g., HEK293T, HEK293F, HEK293FT, HEK293FTM, HEK293SG, HEK293H, HEK293E, HEK293A, or another variant of HEK293)), 293 EBNA, MSR 293, MDCK, HaK, BHK), HeLa, HepG2, WI38, MRCS, Colo205, HB 8065, HL-60, (e.g., BHK21), Jurkat, Daudi, A431 (epidermal), CV-1, U937, 3T3, L cell, C127, SP2/0, NS-0, MMT 060562, Sertoli, BRL 3 A cell, HT1080, myeloma cell, tumor cell, or a cell line derived from an aforementioned cell. Host cells used in methods, compositions, and systems described herein may comprise or be adherent or suspension cells.

“Improve,” “increase,” “inhibit,” or “reduce”: As used herein, the terms “improve”, “increase,” “inhibit,” “reduce,” or grammatical equivalents thereof, indicate values that are relative to a baseline or other reference measurement. In some embodiments, an appropriate reference measurement may be or comprise a measurement in a particular system (e.g., in a single sample, e.g., of a culture medium) under otherwise comparable conditions absent presence of (e.g., prior to and/or after) a particular agent or treatment, or in presence of an appropriate comparable reference agent. In some embodiments, an appropriate reference measurement may be or comprise a measurement in a comparable system known or expected to respond in a particular way, in presence of the relevant agent or treatment.

Medium: As used herein, the terms “medium,” “culture medium,” and “growth medium,” refer to a solution comprising nutrients to nourish cells (e.g., growing cells, e.g., mammalian cells). Typically, these solutions provide essential and non-essential amino acids, vitamins, energy sources, lipids, and/or trace elements required by cells for survival and/or minimal growth. Solutions can also comprise components that enhance survival and/or growth above a minimal rate, such as hormones and growth factors. Solutions can be formulated to a pH and concentration of one or more salts that are optimal for cellular survival and/or proliferation. For example, medium can be a “defined medium” or “chemically defined medium,” e.g., a serum-free medium that contains no proteins, hydrolysates, or components of unknown composition. Defined media are free of animal-derived components and all components have a known chemical structure. One of skill in the art understands defined media can comprise recombinant polypeptides including, but not limited to, hormones, cytokines, interleukins, and/or other signaling molecules.

Microtubule destabilizing agent: As used herein, the term “microtubule destabilizing agent” refers to any agent that interferes directly or indirectly with stability of microtubule polymers and/or microtubule polymerization, thereby inhibiting the physiological function of microtubules. Microtubules are polymers of tubulin and a major component of mitotic spindles, which separate duplicated chromosomes during cell division. If microtubules are destabilized, such as by a microtubule destabilizing agent, cells cannot divide into daughter cells. A microtubule destabilizing agent can arrest cell cycle of cells, such as by depolarizing microtubules, inactivating spindle fiber formation, blocking polymerization of tubules by binding tubulin, depolarizing microtubules by binding tubulin and inducing self-association, and/or inhibiting tubulin formation. A microtubule destabilizing agent can comprise or be a cell cycle arresting agent, e.g., a cell cycle arresting agent described herein. A microtubule destabilizing agent can comprise or be a G2/M inhibitor. A microtubule destabilizing agent can comprise or be colcemid or a derivative or salt thereof. A microtubule destabilizing agent can comprise or be colchicine or a derivative or salt thereof. A microtubule destabilizing agent can comprise or be a Vinca alkaloid (e.g., vinblastine or vincristine or a derivative or salt thereof). A microtubule destabilizing agent can comprise or be vinblastine or a derivative or salt thereof. A microtubule destabilizing agent can comprise or be vincristine or a derivative or salt thereof.

Nucleic acid: As used herein, the term “nucleic acid” includes any nucleotides and analogs or polymers thereof. The term “polynucleotide,” as used herein, refer to a polymeric form of nucleotides of any length, either ribonucleotides (RNA), deoxyribonucleotides (DNA) or a combination thereof. These terms refer to the primary structure of the molecules and, thus, include double-stranded and single-stranded DNA, and double-stranded and single-stranded RNA. These terms include, as equivalents, analogs of either RNA or DNA made from nucleotide analogs and modified polynucleotides including, but not limited to, methylated, protected and/or capped nucleotides or polynucleotides. The terms encompass poly-ribonucleotides or oligo-ribonucleotides (RNA) and poly-deoxyribonucleotides or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to as “internucleotide linkages”). The term encompasses nucleic acids containing any combination of nucleobases, modified nucleobases, sugars, modified sugars, phosphate bridges, or modified phosphorus atom bridges. Examples include, but are not limited to, nucleic acids comprising ribose moieties and/or deoxyribose moieties as well as modified ribose moieties and/or deoxyribose moieties. In some embodiments, the prefix poly- refers to a nucleic acid containing about 2 to about 10,000, about 2 to about 50,000, or about 2 to about 100,000 nucleotide monomer units. In some embodiments, the prefix oligo- refers to a nucleic acid containing about 2 to about 200 nucleotide monomer units. In accordance with methods and compositions described herein, in some embodiments, an RNA comprises or is a microRNA (miRNA), short hairpin RNA (shRNA), small interfering RNA (siRNA), mRNA, snRNA, CRISPR/Cas guide RNA (gRNA), and/or a precursor thereof.

Payload: As used herein, the term “payload” refers to any entity of interest for delivery by a vector. For example, a payload may be desired to be introduced into a cell, organ, organism, and/or biological system (e.g., comprising cells). In some embodiments, a payload sequence is or comprises a heterologous nucleic acid sequence for delivery by a viral vector described herein. In some embodiments, a payload sequence comprises one or more of: an encoding region, a gene regulatory element, and a transcription terminator. Non-limiting examples of gene regulatory elements include promoters, transcriptional activators, enhancers, and polyadenylation signals. In some embodiments, a payload sequence comprises an encoding region, a gene regulatory element, and a transcription terminator, positioned relative to each other such that the encoding region is between gene regulatory element and transcription terminator. In some embodiments, an encoding region encodes a gene product. In some embodiments, a gene product is an RNA. In some embodiments, an encoding region encodes a regulatory RNA (e.g., an miRNA, siRNA, shRNA, mRNA, snRNA, CRISPR/Cas guide RNA (gRNA), zinc finger nuclease, or a precursor thereof). In some embodiments, an encoding region encodes a polypeptide (e.g., an enzyme or antibody). In some embodiments, an encoding region encodes at least one gene product (e.g., 2, 3, 4, 5, 6, 7, or more gene products). One of skill in the art will recognize that a payload can be selected from any heterologous protein or nucleic acid of interest. As used herein, “encode” or “encodes” means directs expression of or processed into. For example, a nucleic acid can encode a polypeptide by directing expression of that polypeptide. As another example, a nucleic acid precursor (e.g., a pri-miRNA or pre-miRNA) can encode a further processed version of a nucleic acid (e.g., mature miRNA) if it is processed into that further processed version.

Polypeptide: The term “polypeptide”, as used herein, refers to a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (e.g., fragments retaining at least one activity) of such complete polypeptides. Moreover, those of ordinary skill in the art understand that protein sequences generally tolerate some substitution without destroying activity. Thus, any polypeptide that retains activity and shares at least about 30% to about 40% overall sequence identity, often greater than about 50%, 60%, 70%, or 80%, and further usually including at least one region of much higher identity, often greater than 90% or even 95%, 96%, 97%, 98%, 99%, or greater in one or more highly conserved regions, usually encompassing at least 3 amino acids to 4 amino acids and often up to 20 amino acids or more, with another polypeptide of the same class, is encompassed within the term “polypeptide” as used herein. Polypeptides may contain L-amino acids, D-amino acids, or both. Polypeptides may contain any of a variety of amino acid modifications or analogs known in the art. Useful modifications include, e.g., terminal acetylation, amidation, and/or methylation. In some embodiments, polypeptides comprise natural amino acids, non-natural amino acids, synthetic amino acids, or combinations thereof. The term “peptide” is generally used to refer to a polypeptide having a length of less than about 100 amino acids, less than about 50 amino acids, less than 20 amino acids, or less than 10 amino acids.

Recombinant AAV (rAAV) particle: A “recombinant AAV particle”, or “rAAV particle,” as used herein, refers to an infectious, replication-defective viral particle comprising an AAV protein shell encapsulating at least one payload that is flanked on both sides by inverted terminal repeats (ITRs) in a vector. An rAAV particle can be produced in suitable host cells described herein (e.g., HEK293 cells, CHO-K cells, HeLa cells, or a variant thereof). For example, host cells are transfected with one or more vectors encoding: at least one payload flanked by an ITR on either side of the at least one payload, at least one Rep polypeptide, at least one Cap polypeptide, and at least one helper polypeptide, such that the host cells are capable of producing Rep, Cap and helper polypeptides necessary for packaging of rAAV particles. rAAV particles described herein may be used for subsequent gene delivery.

Rep polypeptide: The term “Rep polypeptide,” as used herein, refers to AAV non-structural proteins that mediate AAV replication for production of AAV particles. In some embodiments, a Rep polypeptide comprises an AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25 Cap polypeptide, or a variant of any of the foregoing. AAV replication genes and proteins have been described in, e.g., Knipe et al., FIELDS VIROLOGY, Volume 1, (6th ed., Lippincott-Raven Publishers), which is hereby incorporated by reference in its entirety.

Substantially: As used herein, the term “substantially” refers to a qualitative condition of exhibiting total or near-total extent or degree of a characteristic or property of interest. The term “substantially” is therefore used herein to capture a potential lack of absoluteness inherent in many biological and/or chemical effects.

Titer: As used herein, the term “titer” refers to a quantity of virus in a given volume. Titer, for example, can be expressed as viral genome copies (vg) per given volume or plaque forming units (pfu) per given volume. In some embodiments, titer can be expressed as number of capsids per given volume.

Transfection: As used herein, the term “transfection” refers to introduction of nucleic acid sequences, such as DNA or RNA (e.g., mRNA), into cells, such as eukaryotic cells (e.g., mammalian cells). For example, transfection can include vector-based transfection, viral-based transfection, electroporation, lipofection (e.g., with cationic lipids and/or liposomes), calcium phosphate precipitation, nanoparticle-based transfection, and/or transfection based on cationic polymers (e.g., polyethylenimine or DEAE-dextran).

Vector: As used herein, the term “vector” refers to a nucleic acid molecule capable of transporting another nucleic acid to which it has been linked, e.g., into a cell (e.g., a host cell described herein), tissue, and/or organism. By way of non-limiting example, one type of vector is a “viral vector”, in which additional DNA segments may be ligated into a viral genome. Another type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to as “expression vectors.”

Standard techniques may be used for recombinant DNA, oligonucleotide synthesis, and tissue culture and transformation (e.g., electroporation and/or lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification (See, e.g., Sambrook et al., Molecular Cloning: A Laboratory Manual (2d ed., Cold Spring Harbor Laboratory Press, Cold Spring Harbor, N.Y. (1989)), which is incorporated herein by reference in its entirety).

### DETAILED DESCRIPTION

The present disclosure provides, among other things, methods, compositions, and systems for producing a plurality of recombinant viral vector particles (e.g., recombinant adeno-associated virus (rAAV) particles). Generally, rAAV particles produced using methods described herein may be of any AAV serotype known in the art or described herein.

The present disclosure is based, in part, on the discovery that the use of at least one microtubule destabilizing agent surprisingly results in improved production of recombinant viral vector particles (e.g., rAAV particles). In contrast to production of proteins by transient transfection of host cells, which are normal cellular by-products, production of recombinant viral vector particles (e.g., rAAV particles) poses challenges in greater complexity due to the relationship between recombinant viral vectors and host cells (See, e.g., Walsh et al. (2013) Cold Spring Harb Perspect Biol. 2013 5(1), a012351, which is hereby incorporated by reference in its entirety). Recombinant viral vectors depend on cellular machinery for reproduction, while host cells have defense mechanisms against recombinant viral vectors as foreign particles. Further, recombinant viral vectors have mechanisms to inhibit host cell defenses. For example, Rep and Cap polypeptides can be toxic to host cells.

Despite these challenges, the present disclosure provides, inter alia, a recombinant viral vector (e.g., rAAV) production process (e.g., a large-scale manufacturing process) using at least one microtubule destabilizing agent that efficiently generates high titers (e.g., genome titer) of recombinant viral vector particles. In some embodiments, a higher titer of recombinant viral vector particles (e.g., rAAV particles) is relative to recombinant viral vector particles produced under the same conditions and in the same medium, but without at least one microtubule destabilizing agent.

In some embodiments, a higher titer (e.g., genome titer) of recombinant viral vector particles (e.g., rAAV particles) is at least about a 1-fold higher titer, e.g., between about a 1-fold higher titer and about a 10-fold higher titer, about a 2-fold higher titer and about a 3-fold higher titer, about a 3-fold higher titer and about a 4-fold higher titer, about a 1-fold higher titer and about a 5-fold higher titer, about a 3-fold higher titer and about a 4-fold higher titer, about a 4 fold-higher titer and about a 5-fold higher titer, about a 2-fold higher titer and about a 4-fold higher titer, about a 1 fold-higher titer and about a 2-fold higher titer, or about a 1-fold higher titer and about a 4-fold higher titer, e.g., about a 2-fold, about a 3-fold, about a 4-fold, about a 5-fold, about a 6-fold, about a 7-fold, about a 8-fold, about a 9-fold, or about a 10-fold, e.g., relative to recombinant viral vector particles (e.g., rAAV particles) produced under the same conditions and in the same medium, but without a microtubule destabilizing agent.

In some embodiments, a higher titer (e.g., genome titer) of recombinant viral vector particles (e.g., rAAV particles) is greater than about 1.0×10⁹ vg/ml, e.g., between about 1.0×10⁹ vg/ml to about 1.0×10¹² vg/ml, e.g., between about 1.0×10⁹ vg/ml to about 1.0×10¹¹ vg/ml, about 1.0×10¹⁰ vg/ml to about 1.0×10¹² vg/ml, about 1.0×10¹⁰ vg/ml to about 1.0×10¹¹ vg/ml, about 5.0×10⁹ vg/ml to about 1.0×10¹² vg/ml, about 5.0×10⁹ vg/ml to about 1.0×10¹¹ vg/ml, about 5.0×10¹⁰ vg/ml to about 1.0×10¹² vg/ml, about 1.0×10⁹ vg/ml to about 5.0×10¹¹ vg/ml, or about 1.0×10¹⁰ vg/ml to about 5.0×10¹¹ vg/ml, e.g., when cultured in a bioreactor (e.g., a batch bioreactor or a fed batch bioreactor, e.g., at manufacturing scale (e.g., about 250 liters volume)).

Microtubule Destabilizing Agents

The present disclosure, among other things, provides microtubule destabilizing agents for producing recombinant viral vector particles (e.g., rAAV particles). Microtubules are polymers of tubulin and a major component of mitotic spindles, which separate duplicated chromosomes during cell division. Many forms of microtubule destabilizing agents can be used in methods, compositions, and systems described herein for producing recombinant viral vector particles (e.g., rAAV particles). In some embodiments, a microtubule destabilizing agent described herein depolarizes microtubules, inactivates spindle fiber formation, blocks polymerization of tubules by binding tubulin, induces tubulin self-association, and/or inhibits tubulin formation. In some embodiments, a microtubule destabilizing agent comprises one, two, three, or more (e.g., all) of colcemid, colchicine, vinblastine, or vincristine, or a derivative or salt thereof.

In some embodiments, a microtubule destabilizing agent comprises or is colcemid or a derivative or salt thereof. Colcemid (also known as demecolcine, N-methyl-N-deacetyl-colchicine, and N-deacetyl-N-methylcolchicine) has the following general formula:

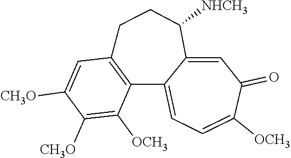

In some embodiments, colcemid or a derivative or salt thereof arrests cell cycle of host cells described herein. In some embodiments, colcemid or a derivative or salt thereof depolarizes microtubules and/or inactivates spindle fiber formation. In some embodiments, colcemid or a derivative or salt thereof arrests cell cycle of host cells described herein at G2/M phase. In some embodiments, colcemid or a derivative or salt thereof comprises or is a G2/M inhibitor.

In some embodiments, a microtubule destabilizing agent described herein (e.g., colcemid or a derivative or salt thereof) is added to medium comprising host cells described herein at a concentration of about 0.1 μM to about 3.0 μM, about 0.2 μM to about 2.5 μM, about 0.3 μM to about 2.0 μM, about 0.4 μM to about 1.5 μM, about 0.1 μM to about 1.0 μM, about 0.2 μM to about 1.4 μM, about 0.3 μM to about 1.8 μM, about 0.4 μM to about 1.2 μM, or about 0.1 μM to about 1.4 μM, e.g., about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, about 2.5 μM, about 2.6 μM, about 2.7 μM, about 2.8 μM, about 2.9 μM, or about 3.0 μM.

In some embodiments, a microtubule destabilizing agent comprises or is colchicine or a derivative or salt thereof. Colchicine (also known as (S)—N-(5,6,7,9-tetrahydro-1,2,3,10-tetramethoxy-9-oxobenzo[a]heptalen-7-yl)acetamide) has the following general formula:

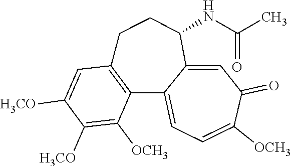

In some embodiments, colchicine or a derivative or salt thereof arrests cell cycle of host cells described herein. In some embodiments, colchicine or a derivative or salt thereof blocks polymerization of tubules by binding tubulin. In some embodiments, colchicine or a derivative or salt thereof arrests cell cycle of host cells described herein at G2/M phase. In some embodiments, colchicine or a derivative or salt thereof comprises or is a G2/M inhibitor.

In some embodiments, a microtubule destabilizing agent described herein (e.g., colchicine or a derivative or salt thereof) is added to medium comprising host cells described herein at a concentration of about 1 μM to about 25 μM, about 2 μM to about 20 μM, about 3 μM to about 15 μM, about 4 μM to about 10 μM, or about 5 μM to about 15 μM, about 1 μM to about 8 μM, about 2 μM to about 14 μM, about 3 μM to about 12 μM, about 4 μM to about 22 μM, or about 5 μM to about 25 μM, e.g., about 1 μM, about 1.5 μM, about 2 μM, about 2.5 μM, about 3.0 μM, about 3.5 μM, about 4.0 μM, about 4.5 μM, about 5.0 μM, about 5.5 μM, about 6.0 μM, about 6.5 μM, about 7.0 μM, about 7.5 μM, about 8.0 μM, about 8.5 μM, about 9.0 μM, about 9.5 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, or about 25 μM.

In some embodiments, a microtubule destabilizing agent comprises or is a Vinca alkaloid (e.g., vinblastine or vincristine or a derivative or salt thereof). In some embodiments, a microtubule destabilizing agent comprises or is vinblastine or a derivative or salt thereof. In certain embodiments, a microtubule destabilizing agent comprises or is vinblastine sulfate. Vinblastine (also known as vincaleukoblastine) has the following general formula:

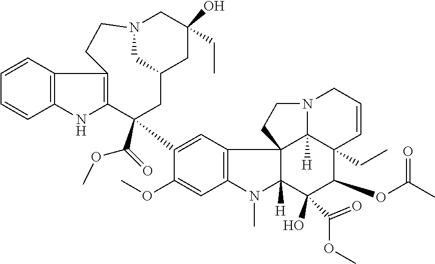

In some embodiments, vinblastine or a derivative or salt thereof arrests cell cycle of host cells described herein. In some embodiments, vinblastine or a derivative or salt thereof depolarizes microtubules by binding tubulin and/or induces self-association. In some embodiments, vinblastine or a derivative or salt thereof arrests cell cycle of host cells described herein at G2/M phase. In some embodiments, vinblastine or a derivative or salt thereof comprises or is a G2/M inhibitor.

In some embodiments, a microtubule destabilizing agent described herein (e.g., vinblastine or a derivative or salt thereof) is added to medium comprising host cells described herein at a concentration of about 0.2 μM to about 10 μM, about 0.4 μM to about 2.0 μM, about 0.6 μM to about 4.0 μM, about 0.8 μM to about 6.0 μM, about 1.0 μM to about 8.0 μM, about 0.4 μM to about 7.0 μM, about 0.6 μM to about 6.0 μM, about 0.8 μM to about 5.0 μM, or about 1.0 μM to about 3.0 μM, e.g., about 0.2 μM, about 0.3 μM, about 0.4 μM, about 0.5 μM, about 0.6 μM, about 0.7 μM, about 0.8 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, about 2.5 μM, about 2.6 μM, about 2.7 μM, about 2.8 μM, about 2.9 μM, about 3.0 μM, about 3.5 μM, about 4.0 μM, about 4.5 μM, about 5.0 μM, about 5.5 μM, about 6.0 μM, about 6.5 μM, about 7.0 μM, about 7.5 μM, about 8.0 μM, about 8.5 μM, about 9.0 μM, about 9.5 μM, or about 10 μM.

In some embodiments, a microtubule destabilizing agent comprises or is vincristine or a derivative or salt thereof. In certain embodiments, a microtubule destabilizing agent comprises or is vincristine sulfate. Vincristine (also known as leurocristine) has the following general formula:

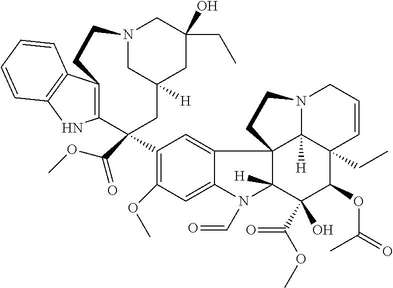

In some embodiments, vincristine or a derivative or salt thereof arrests cell cycle of host cells described herein. In some embodiments, vincristine or a derivative or salt thereof inhibits tubulin formation. In some embodiments, vincristine or a derivative or salt thereof arrests cell cycle of host cells described herein at G2/M phase. In some embodiments, vincristine or a derivative or salt thereof comprises or is a G2/M inhibitor.

In some embodiments, a microtubule destabilizing agent described herein (e.g., vincristine or a derivative or salt thereof) is added to medium comprising host cells described herein at a concentration of about 0.2 μM to about 10 μM, about 0.4 μM to about 2.0 μM, about 0.6 μM to about 4.0 μM, about 0.8 μM to about 6.0 μM, about 1.0 μM to about 8.0 μM, about 0.4 μM to about 7.0 μM, about 0.6 μM to about 6.0 μM, about 0.8 μM to about 5.0 μM, or about 1.0 μM to about 3.0 μM, e.g., about 0.2 μM, about 0.3 μM, about 0.4 μM, about 0.5 μM, about 0.6 μM, about 0.7 μM, about 0.8 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, about 2.5 μM, about 2.6 μM, about 2.7 μM, about 2.8 μM, about 2.9 μM, about 3.0 μM, about 3.5 μM, about 4.0 μM, about 4.5 μM, about 5.0 μM, about 5.5 μM, about 6.0 μM, about 6.5 μM, about 7.0 μM, about 7.5 μM, about 8.0 μM, about 8.5 μM, about 9.0 μM, about 9.5 μM, or about 10 μM.

In some embodiments, a microtubule destabilizing agent is added to medium comprising host cells substantially simultaneously with one or more vectors described herein. In some embodiments, a microtubule destabilizing agent is added to medium comprising host cells within one minute of transfection, e.g., within about 55 seconds, about 50 seconds, about 45 seconds, seconds, about 40 seconds, about 35 seconds, about 30 seconds, about 25 seconds, about 20 seconds, about 15 seconds, about 10 seconds, about 5 seconds, or less before or after transfection.

In some embodiments, a microtubule destabilizing agent is added to medium comprising host cells prior to transfection with one or more vectors described herein. In some embodiments, an antioxidant is added to medium during preparation, i.e., before the medium is contacted with the host cells. In some embodiments, a microtubule destabilizing agent is added about 24 hours to about 1 minute, about 12 hours to about 20 minutes, about 6 hours to about 10 minutes, about 4 hours to about 30 minutes, about 2 hours to about 15 minutes, about 1 hour to about 10 minutes, about 50 minutes to about 15 minutes, about 45 minutes to about 20 minutes, about 50 minutes to about 20 minutes, or about 35 minutes to about 15 minutes prior to transfection, e.g., about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, about 1 hour, about 50 minutes, about 40 minutes, about 30 minutes, about 20 minutes, about 10 minutes, about 5 minutes, about 4 minutes, about 3 minutes, about 2 minutes, or about 1 minute prior to transfection.

In some embodiments, a microtubule destabilizing agent is added to medium comprising host cells after transfection with one or more vectors described herein. In some embodiments, a microtubule destabilizing agent is added about 24 hours to about 1 minute, about 12 hours to about 20 minutes, about 6 hours to about 10 minutes, about 4 hours to about 30 minutes, about 2 hours to about 15 minutes, about 1 hour to about 10 minutes, about 50 minutes to about 15 minutes, about 45 minutes to about 20 minutes, about 50 minutes to about 20 minutes, or about 35 minutes to about 15 minutes after transfection, e.g., about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, about 1 hour, about 50 minutes, about 40 minutes, about 30 minutes, about 20 minutes, about 10 minutes, about 5 minutes, about 4 minutes, about 3 minutes, about 2 minutes, or about 1 minute after transfection.

Transfection

The present disclosure, among other things, provides methods for transfection of host cells with one or more vectors described herein for producing recombinant viral vector particles (e.g., rAAV particles). Host cells (e.g., mammalian cells, such as HEK293 cells, CHO-K cells, HeLa cells, or a variant of thereof) can be transfected with one or more vectors encoding: (i) at least one payload (e.g., an inhibitory nucleic acid or a polypeptide as described herein) flanked by an inverted terminal repeat (ITR) on either side of the at least one payload, (ii) at least one Rep polypeptide, (iii) at least one Cap polypeptide, and (vii) at least one helper polypeptide. In some embodiments, transfection comprises or is transient transfection. In some embodiments, transfection comprises or is stable transfection. In some embodiments, host cells are transfected with two or three vectors described herein.

Transfection can include any method known to a skilled person for introducing one or more exogenous nucleic acids into host cells (e.g., mammalian cells, such as HEK293 cells, CHO-K cells, HeLa cells, or a variant of thereof). For example, transfection can include, but is not limited to, vector-based transfection (e.g., plasmid-based transfection), viral-based transfection, electroporation, lipofection (e.g., with one or more cationic lipids and/or liposomes), calcium phosphate precipitation, nanoparticle-based transfection, transfection based on cationic polymers (e.g., DEAE-dextran and/or polyethylenimine), or a combination of any of the foregoing. Various transfection techniques are generally known in those skilled in the art (See, e.g., Graham et al. (1973) Virology, 52:456; Sambrook et al. (1989) Molecular Cloning, a laboratory manual, Cold Spring Harbor Laboratories; New York, Davis et al. (1986) Basic Methods in Molecular Biology, Elsevier; and Chu et al. (1981) Gene 13:197, each of which is hereby incorporated by reference in its entirety).

In some embodiments, host cells (e.g., mammalian cells, such as HEK293 cells, CHO-K cells, HeLa cells, or a variant of thereof) are transfected with a transfection reagent. In some embodiments, a transfection reagent comprises or is a polymer. In some embodiments, a transfection reagent comprises or is polyethylenimine (PEI)), calcium phosphate, a lipid capable of traversing a cell membrane (e.g., a liposome or a micelle), a nanoparticle, or a combination thereof. In certain embodiments, a transfection reagent comprises or is PEI.

Host Cells

The present disclosure, among other things, provides host cells for producing recombinant viral vector particles (e.g., rAAV particles). A host cell includes a progeny cell of an original cell transfected with at least one vector described herein. A progeny cell of a parental cell may not be substantially identical in morphology or genomic content as a parent cell due to natural, accidental, and/or deliberate mutation.

One or more components for a host cell to produce recombinant viral vector particles (e.g., rAAV particles) may be provided in trans on at least one vector. A stable host cell may comprise at least one polypeptide to produce recombinant viral vector particles (e.g., rAAV particles) using methods known to those of skill in the art. In some embodiments, a stable host cell comprises at least one polypeptide under control of an inducible promoter. In other embodiments, a stable host cell comprises at least one polypeptide under control of a constitutive promoter. For example, a stable host cell (e.g., a HEK293 cell or a variant thereof) may comprise a nucleic acid encoding an E1 helper polypeptide under the control of a constitutive promoter. Other stable host cells may be generated by one of skill in the art using routine methods.

Exemplary host cells include, but are not limited to, prokaryotes or eukaryotes (single or multiple cell), bacterial cells (e.g., strains of E. coli, Bacillus spp., or Streptomyces spp.), mycobacteria cells, fungal cells, yeast cells (e.g., S. cerevisiae, S. pombe, P. pastoris, or P. methanolica), plant cells, insect cells (e.g., SF-9, SF-21, baculovirus-infected insect cells, or Trichoplusia ni), non-human animal cells, human cells, or cell fusions (e.g., hybridomas or quadromas). In some embodiments, a host cell comprises or is a mammalian cell. In some embodiments, a mammalian cell comprises or is a human cell, monkey cell, ape cell, hamster cell, rat cell, or mouse cell.

In some embodiments, a host cell comprises or is a kidney cell (e.g., HEK293, 293 EBNA, MSR 293, MDCK, HaK, BHK, or a variant thereof). In some embodiments, a host cell comprises or is a HEK293 cell or a variant thereof. In certain embodiments, a host cell comprises or is HEK293T, HEK293F, HEK293FT, HEK293FTM, HEK293SG, HEK293H, HEK293E, HEK293A, or a variant thereof (See, e.g., Yuan et al. Biomed Pharmacol 2018; 11 (2), which is hereby incorporated by reference in its entirety). In certain embodiments, a HEK293 cell or a variant thereof comprises or expresses an E1 polypeptide. In some embodiments, a host cell comprises or is a CHO cell (e.g., CHO K1, DXB-11 CHO, Veggie-CHO, or a variant thereof). In certain embodiments, a host cell comprises or is a HeLa cell or a variant thereof.

In some embodiments, a host cell comprises or is a COS cell (e.g., COS-7 or a variant thereof). In some embodiments, a host cell comprises or is a retinal cell. In some embodiments, a host cell comprises or is a Vero cell. In some embodiments, a host cell comprises or is a CV1 cell. In some embodiments, a host cell comprises or is a HepG2 cell. In some embodiments, a host cell comprises or is a WI38 cell. In some embodiments, a host cell comprises or is a MRCS cell. In some embodiments, a host cell comprises or is a Colo205 cell. In some embodiments, a host cell comprises or is a HB 8065 cell. In some embodiments, a host cell comprises or is a HL-60 cell (e.g., BHK21 or a variant thereof). In some embodiments, a host cell comprises or is a Jurkat cell. In some embodiments, a host cell comprises or is a Daudi cell. In some embodiments, a host cell comprises or is an A431 epidermal cell. In some embodiments, a host cell comprises or is a CV-1 cell. In some embodiments, a host cell comprises or is a U937 cell. In some embodiments, a host cell comprises or is a 3T3 cell. In some embodiments, a host cell comprises or is an L cell. In some embodiments, a host cell comprises or is a C127 cell. In some embodiments, a host cell comprises or is a SP2/0 cell. In some embodiments, a host cell comprises or is a NS-0 cell. In some embodiments, a host cell comprises or is a MMT060562 cell. In some embodiments, a host cell comprises or is a Sertoli cell. In some embodiments, a host cell comprises or is a BRL3A cell. In some embodiments, a host cell comprises or is a HT1080 cell. In some embodiments, a host cell comprises or is a myeloma cell. In some embodiments, a host cell comprises or is a tumor cell. In some embodiments, a host cell comprises or is a cell line or variant derived from any cell described herein.

In some embodiments, prior to transfection, host cells (e.g., adherent or suspended host cells) are seeded at a certain density. The term “seeding,” as used herein, refers to the process of providing a cell culture to a vessel (e.g., a bioreactor or culture flask). For example, the process of providing a cell culture may include propagation of host cells in another bioreactor or vessel before providing host cells to the bioreactor or other vessel. Host cells may be frozen and thawed immediately prior to providing to a bioreactor or vessel.

In some embodiments, host cells comprise or are adherent cells. In some embodiments, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or more host cells in culture are adherent cells.

In some embodiments, host cells comprise or are suspension cells. In some embodiments, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or more host cells in culture are suspension cells.

Vectors

The present disclosure, among other things, provides vectors for producing recombinant viral vector particles (e.g., rAAV particles). Many forms of vectors can be used in methods, compositions, and systems described herein for producing recombinant viral vector particles (e.g., rAAV particles). Non-limiting examples of vectors for producing recombinant viral vector particles (e.g., rAAV particles) include, but are not limited to, plasmids, bacteriophage vectors, cosmids, phagemids, and artificial chromosomes.

In some embodiments, a vector encodes at least one payload flanked by an inverted terminal repeat (ITR) on either side of the at least one payload (e.g., for expression of an inhibitory nucleic acid or polypeptide described herein). In some embodiments, a vector encodes at least one Rep polypeptide. In some embodiments, a vector encodes least one Cap polypeptide. In some embodiments, a vector encodes at least one helper polypeptide.

In some embodiments, a vector encodes at least one Rep polypeptide and at least one Cap polypeptide. In some embodiments, a vector encodes at least one Cap polypeptide and at least one payload flanked by an ITR on either side of the at least one payload. In some embodiments, a vector encodes at least one helper polypeptide and at least one Rep polypeptide. In some embodiments, a vector encodes at least one helper polypeptide and at least one payload flanked by an ITR on either side of the at least one payload. In some embodiments, a vector encodes at least one Rep polypeptide and at least one payload flanked by an ITR on either side of the at least one payload. In some embodiments, a vector encodes at least one helper polypeptide and at least one Cap polypeptide.

In some embodiments, methods described herein comprise transfecting a host cell with at least three vectors. In some embodiments, three vectors comprise: (i) a first vector encoding at least one payload flanked by an ITR on either side of the at least one payload, (ii) a second vector encoding at least one Rep polypeptide and at least one Cap polypeptide, and (iii) a third vector encoding at least one helper polypeptide. In some embodiments, methods described herein comprises transfecting a host cell with at least two vectors. In some embodiments, at least two vectors comprise: (i) a first vector encoding at least one Cap polypeptide and at least one payload flanked by an ITR on either side of the at least one payload; and (ii) a second vector encoding at least one helper polypeptide and at least one Rep polypeptide.

A vector can include conventional control elements operably linked to a nucleic acid encoding any payload or polypeptide described herein, in a manner that permits transcription, translation and/or expression in a cell transfected with a vector. Expression control sequences include, but are not limited to, transcription, initiation, termination, promoter, and/or enhancer sequences; efficient RNA processing signals, such as splicing and/or polyadenylation (polyA) signals (e.g., a rabbit β-globin polyA signal); sequences that stabilize cytoplasmic mRNA; sequences that enhance translation efficiency (e.g., a Kozak consensus sequence); sequences that enhance protein stability; and when desired, sequences that enhance secretion of an encoded product. In some embodiments, a vector includes other regulatory elements, such as at least one Woodchuck Hepatitis Virus (WHP) Posttranscriptional Regulatory Element (WPRE).

A number of expression control sequences, including promoters that are native, constitutive, inducible, and/or tissue-specific, are known in the art and may be included in a vector described herein. Examples of constitutive promoters include, but are not limited to, a retroviral Rous sarcoma virus (RSV) LTR promoter (e.g., further comprising an RSV enhancer), a cytomegalovirus (CMV) promoter (e.g., further comprising an CMV enhancer), an SV40 promoter, and an dihydrofolate reductase promoter.

Inducible promoters allow regulation of gene expression and can be regulated by exogenously supplied compounds, environmental factors, such as temperature, or the presence of a specific physiological state (e.g., acute phase, a particular differentiation state of the cell, or in replicating cells only). Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system, an ecdysone insect promoter, a tetracycline-repressible system, a tetracycline-inducible system, a RU486-inducible system, and an rapamycin-inducible system. Still other types of inducible promoters that may be useful are regulated by a specific physiological state, such as temperature, acute phase, a particular differentiation state of the cell, or in replicating cells only.

In some embodiments, a native promoter or fragment thereof for a nucleic acid encoding any payload or polypeptide described herein may be used. Other native expression control elements, such as enhancer elements, polyadenylation sites, or Kozak consensus sequences, may also be used to mimic native expression.

Vectors Encoding at Least One Payload

The present disclosure, among other things, provides vectors (e.g., plasmids) encoding at least one payload. A payload sequence is generally a sequence of interest to be introduced into a cell, tissue, organ, or organism. An rAAV vector comprises cis-acting 5′ and 3′ inverted terminal repeat (ITR) sequences (See, e.g., B. J. Carter, in “Handbook of Parvoviruses,” ed., P. Tijsser, CRC Press, pp. 155-168 (1990), which is hereby incorporated by reference in its entirety).

As used herein, the terms “5″” and “3″” are relative terms to define a spatial relationship or directionality between two or more segment of a nucleic acid sequence. Thus, 3′ of a nucleic acid indicates a segment of the nucleic acid that is downstream of another segment, while 5′ indicates a segment of the nucleic acid that is upstream of another segment. For example, 3′ may indicate that a segment is in the 3′ half of the nucleic acid sequence or even at the 3′ end of the nucleic acid sequence. Similarly, 5′ may indicate that a segment is in the 5′ half of the nucleic acid sequence or even at the 5′ end of the nucleic acid sequence. Unless indicated otherwise, the directionality of a nucleic acid will be in the 5′ to 3′ direction of translation.

ITR sequences are typically about 115 to 145 nt in length. In some embodiments, one or both of a 5′ ITR or a 3′ ITR nucleic acid sequence are modified via insertion, deletion and/or substitution relative to a known ITR nucleic acid sequence, e.g., the 145 nt wild-type 5′ and 3′ ITRs from an AAV2 genome. Modification of ITR nucleic acid sequences is known to those of skill in the art (See, e.g., Sambrook et al, “Molecular Cloning. A Laboratory Manual”, 2d ed., Cold Spring Harbor Laboratory, New York (1989); and K. Fisher et al., J Virol., 70:520 532 (1996), each of which is hereby incorporated by reference in its entirety). AAV ITR sequences may be obtained from any known AAV, including mammalian AAV types, e.g., an AAV2 genome.

In some embodiments, a payload comprises or is a heterologous nucleic acid with a therapeutic purpose, e.g., an miRNA, siRNA, shRNA, mRNA, snRNA, CRISPR/Cas guide RNA (gRNA), or a precursor thereof. In some embodiments, a payload comprises or is a heterologous protein with a therapeutic purpose, e.g., an enzyme, cytokine, antibody, receptor, fusion protein, or chimeric polypeptide.

In some embodiments, a payload is linked to a secretion signal sequence for secretion of an expressed polypeptide. One of skill in the art will recognize that a payload can be selected from any heterologous polypeptide or nucleic acid of interest. In some embodiments, a payload sequence comprises one or more aptamer-binding domains or polypeptide-binding domains (e.g., transcription factor-binding domains). A vector will also typically include other regulatory elements (e.g., promoters, introns, and/or enhancers) to regulate expression or amount of a payload in a cell or tissue.

In some embodiments, a payload sequence can be of any length, e.g., between about 2 nucleotides and about 10,000 nucleotides in length or any integer value there between. In some embodiments, a nucleic acid sequence encoding a payload comprises at least 20 nucleotides, at least 50 nucleotides, at least 75 nucleotides, at least 100 nucleotides, at least 150 nucleotides, at least 200 nucleotides, at least 250 nucleotides, at least 300 nucleotides, at least 350 nucleotides, at least 400 nucleotides, at least 450 nucleotides, at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 900 nucleotides, at least 950 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 2000 nucleotides, at least 2500 nucleotides, at least 3000 nucleotides, at least 4000 nucleotides, at least 5000 nucleotides, at least 6000 nucleotides, at least 7000 nucleotides, at least 8000 nucleotides, or at least 9000 nucleotides. In some embodiments, a nucleic acid sequence encoding a payload comprises between about 50 and 500 nucleotides in length, between about 100 and 1,000 nucleotides in length, between about 250 and 2,000 nucleotides in length, between about 150 and 3,000 nucleotides in length, between about 500 and 6,000 nucleotides in length, between about 700 and 7,000 nucleotides in length, between about 1,000 and 8,000 nucleotides in length, or between about 2,000 and 5,000 nucleotides in length.

An exemplary payload of interest comprises at least one inhibitory nucleic acid (e.g., at least one miRNA, siRNA, shRNA, gRNA, or any combination thereof) that inhibits expression of a protein. In some embodiments, a vector comprises one or more nucleic acids encoding at least one miRNA that inhibits expression of a protein. In some embodiments, a target nucleic acid sequence encoding a protein comprises or is a wild-type nucleic acid sequence, or a mutant or variant thereof. In some embodiments, targeted nucleic acid sequences include or are mRNA sequences. In some embodiments, targeted mRNA sequences comprise or are human mRNA.

In some embodiments, a payload comprises or is a polypeptide with a therapeutic purpose. In some embodiments, a vector comprises one or more nucleic acids for insertion of a nucleic acid sequence encoding a polypeptide with a therapeutic purpose into a genome of a subject (e.g., a human), e.g., for long-term expression of a polypeptide in a subject. In some embodiments, a subject has reduced expression of a polypeptide relative to a subject with a corresponding wildtype gene. In some embodiments, a subject has a mutant polypeptide relative to a subject with a corresponding wildtype gene. In some embodiments, a subject has a dysfunctional polypeptide relative to a subject with a corresponding wildtype gene.

Vectors Encoding at Least One Rep Polypeptide

The present disclosure, among other things, provides vectors (e.g., plasmids) encoding at least one Rep polypeptide. Rep polypeptides are involved in viral DNA replication, resolution of replicative intermediates, and generation of single-stranded genomes. In some embodiments, a vector comprises a nucleic acid sequence encoding one, two, three, or more (e.g., all) of Rep78, Rep68, Rep52, or Rep40, or a variant thereof.

In some embodiments, a Rep polypeptide comprises a nucleic acid sequence derived from AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25 serotype, or a variant of any of the foregoing. For example, a nucleic acid sequence encoding a Rep polypeptide may be derived from a known AAV genome sequence including, but not limited to, AAV1 Accession No. NC_002077 or AF063497; AAV2 Accession No. NC_001401; AAV3A Accession No. NC_001729; AAV3B Accession No. NC_001863; AAV4 Accession No. NC_001829; AAV5 Accession No. Y18065 or AF085716; Accession No. AAV6 NC_001862; Avian AAV ATCC VR-865 AY186198, AY629583, or NC_004828; Avian AAV strain DA-1 NC_006263, AY629583; or Bovine AAV NC_005889, AY388617.

In certain embodiments, a nucleic acid sequence encoding a Rep polypeptide is derived from an AAV genome sequence or a variant thereof as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

In some embodiments, a promoter is operably linked to a nucleic acid sequence encoding at least one Rep polypeptide. In some embodiments, a wild-type promoter of AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, or a variant of any of the foregoing is operably linked to a nucleic acid sequence encoding at least one Rep polypeptide. In certain embodiments, a promoter operably linked to a nucleic acid sequence encoding at least one Rep polypeptide comprises a p5 and/or p19 promoter.

Vectors Encoding at Least One Cap Polypeptide

The present disclosure, among other things, provides vectors (e.g., plasmids) encoding at least one Cap polypeptide. Cap polypeptides (e.g., VP1, VP2, and VP3) are structural polypeptides that form a capsid. In some embodiments, a vector comprises a nucleic acid sequence encoding one, two, or three of VP1, VP2, and VP3.

In some embodiments, a Cap polypeptide comprises a nucleic acid sequence derived from an AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25 serotype, or a variant of any of the foregoing. For example, a nucleic acid sequence encoding a Cap polypeptide may be derived from a known AAV genome sequence including, but not limited to, AAV1 Accession No. NC_002077 or AF063497; AAV2 Accession No. NC_001401; AAV3A Accession No. NC_001729; AAV3B Accession No. NC_001863; AAV4 Accession No. NC_001829; AAV5 Accession No. Y18065 or AF085716; Accession No. AAV6 NC_001862; Avian AAV ATCC VR-865 AY186198, AY629583, or NC_004828; Avian AAV strain DA-1 NC_006263, AY629583; or Bovine AAV NC_005889, AY388617.

In certain embodiments, a nucleic acid sequence encoding a Cap polypeptide is derived from an AAV genome sequence or a variant thereof as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

In some embodiments, a capsid comprises or is a modified capsid protein (e.g., a capsid comprising a modified VP3 region). Methods of producing modified capsid proteins are known in the art (See, e.g., US20130310443, which is hereby incorporated by reference in its entirety). In some embodiments, a modified capsid protein comprises at least one non-native amino acid substitution at a position that corresponds to a surface-exposed amino acid (e.g., a surface exposed tyrosine) in a wild-type capsid protein. In some embodiments, a modified capsid protein comprises a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in a wild-type capsid protein, a non-lysine amino acid (e.g., a glutamic acid) at a position that corresponds to a surface-exposed lysine amino acid in a wild-type capsid protein, a non-serine amino acid (e.g., a valine) at a position that corresponds to a surface-exposed serine amino acid in a wild-type capsid protein, or a combination thereof. In some embodiments, a modified capsid protein comprises or has at least 1, 2, 3, 4, 5, 6, 7, 8, 9, 10, or more amino acid substitutions.

In some embodiments, a promoter is operably linked to a nucleic acid sequence encoding at least one Cap polypeptide. In some embodiments, a wild-type promoter of AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, or a variant of any of the foregoing is operably linked to a nucleic acid sequence encoding at least one Rep polypeptide. In certain embodiments, a p40 promoter is operably linked to a nucleic acid sequence encoding at least one Cap polypeptide.

Vectors Encoding at Least One Helper Polypeptide

The present disclosure, among other things, provides vectors (e.g., plasmids) encoding at least one helper polypeptide. AAV is a helper-dependent DNA parvovirus, which belongs to the genus Dependovirus. Production of recombination AAV requires co-infection with a related virus (e.g., adenovirus, herpes, or vaccinia virus) or a helper vector encoding helper polypeptides, such as structural proteins and proteins for viral genome replication.

A vector encoding at least one helper polypeptide can comprise nucleotide sequences for non-AAV derived viral and/or cellular functions upon which AAV is dependent for replication, which may include, but are not limited to, activation of gene transcription, stage-specific mRNA splicing, DNA replication, synthesis of at least one Cap polypeptide, and/or capsid assembly. Viral-based helper polypeptides can be derived from any known helper virus, such as adenovirus, herpesvirus, vaccinia virus, or a combination thereof. Thus, a vector (e.g., a plasmid) encoding at least one helper polypeptide can comprise sufficient helper function to permit packaging of an rAAV vector into capsid polypeptides. In some embodiments, a helper polypeptide comprises an Ad5 helper polypeptide. In certain embodiments, a nucleic acid sequence of an Ad5 helper polypeptide is derived from Adenovirus 5 genome (GenBank Accession No. AY601635). In some embodiments, a helper polypeptide comprises an Ad2 helper polypeptide.

Helper polypeptides (e.g., Ad5 or Ad2 helper polypeptides) can comprise at least one, two, three, or more (e.g., all) of E1, E2A, E4orf6, or VA RNA. In some embodiments, E1 comprises E1a and/or E1b. In some embodiments, one or both of E2A and VA RNA increase stability and/or efficiency of AAV mRNA translation, such as for cap gene transcripts. In some embodiments, E4orf6 facilitates DNA replication. In some embodiments, E1a comprises a transactivator (e.g., regulating activity of at least one Ad gene, AAV rep gene, and/or AAV cap gene). In some embodiments, E1b comprises a viral mRNA transport. Helper polypeptides are described in further detail in Coura and Nardi, Genetics and and Molecular Biology, 31(1): 1-11 (2008), which is hereby incorporated by reference in its entirety.

Culturing

The present disclosure, among other things, provides methods for culturing host cells described herein for producing recombinant viral vector particles (e.g., rAAV particles). A wide variety of growth media (e.g., mammalian growth media) may be used in methods, compositions, and systems described herein for producing a plurality of recombinant viral vector particles (e.g., rAAV particles). Host cells may be grown in a variety of chemically defined media, in which media components are known and controlled. Cells may also be grown in a complex medium, in which not all media components are known and/or controlled.

Cultures of host cells can be prepared in any medium suitable for a particular host cell type being cultured. Exemplary components of host cell medium can include, but are not limited to, inorganic salts, carbohydrates (e.g., sugars, such as glucose, galactose, maltose, or fructose), amino acids, vitamins (e.g., B group vitamins (e.g., B12), vitamin A, vitamin E, riboflavin, thiamine, or biotin), fatty acids (e.g., cholesterol or steroids), proteins (e.g., albumin, transferrin, fibronectin, or fetuin), serum (e.g., albumins, growth factors, or growth inhibitors, such as fetal bovine serum, newborn calf serum, or horse serum), trace elements (e.g., zinc, copper, selenium, or tricarboxylic acid intermediates), hydrolysates (e.g., derived from plant or animal sources), or combinations thereof.

Commercially available media can be used for culturing host cells described herein. Exemplary media can include, but is not limited to, Dulbecco's Modified Eagle's Medium (DMEM, Sigma), FreeStyle™ F17 Expression Medium (ThermoFisher), DMEM/F12 medium (Invitrogen), CD OptiCHO™ medium (Invitrogen), CD EfficientFeed™ media (Invitrogen), Cell Boost (HyClone™) media (GE Life Sciences), BalanCD™ CHO Feed (Irvine Scientific), BD Recharge™ (Becton Dickinson), Cellvento Feed™ (EMD Millipore), Ex-cell CHOZN Feed™ (Sigma-Aldrich), CHO Feed Bioreactor Supplement (Sigma-Aldrich), SheffCHOT™ (Kerry), Zap-CHO™ (Invitria), ActiCHO™ (PAA/GE Healthcare), Minimal Essential Medium (Sigma), or RPMI-1640 (Sigma).

Media can be supplemented as necessary with hormones and/or other growth factors (e.g., insulin, transferrin, or epidermal growth factor), salts (e.g., sodium chloride, calcium, magnesium, or phosphate), buffers (e.g., HEPES), nucleosides (e.g., adenosine or thymidine), antibiotics (e.g., kanamycin, puromycin, neomycin, hygromycin, blasticidin, gentamycin, Gr18, or zeocin), trace elements, lipids (e.g., linoleic or other fatty acids), or glucose or an equivalent energy source. In some embodiments, media for culturing host cells described herein comprises glutamine or a glutamine dipeptide. In some embodiments, media for culturing host cells described herein comprises a surfactant. In some embodiments, media comprises or is serum-free media, protein-free media, or chemically defined media. Any other necessary supplements can also be included at appropriate concentrations that would be known to those skilled in the art.

After culturing of host cells described herein, a plurality of recombinant viral vector particles (e.g., rAAV particles) can be recovered from culture media. In some embodiments, recombinant viral vector particles (e.g., rAAV particles) are recovered by lysing host cells and recovering recombinant viral vector particles from lysate, e.g., after centrifugation. In some embodiments, recombinant viral vector particles (e.g., rAAV particles) are recovered from culture supernatant. In some embodiments, a lysis solution for host cells comprises chemical reagents, such as detergents (e.g., sodium dodecyl sulfate (SDS), ethyl trimethyl ammonium bromide, Triton X-100, bile salts (e.g., cholate) or zwitterionic detergents (e.g., CHAPS).

In some embodiments, a lysis solution for host cells comprises a salt (e.g., NaCl) and a high pH (e.g., a pH of greater than about 7). In some embodiments, recombinant viral vector particles (e.g., rAAV particles) are purified using purification methods, such as chromatography (e.g., affinity chromatography or ion-exchange chromatography (e.g., cation exchange chromatography)) or filtration (e.g., UF/DF filtration)).

Host cells described herein can be cultured in a cell culture vessel or bioreactor. Cell culture vessels can include cell culture dishes, plates, or flasks. In some embodiments, a cell culture vessel is used for culturing adherent cells. In other embodiments, a cell culture vessel is used for culturing suspension cells. Exemplary cell culture vessels include, but are not limited to, 35 mm, 60 mm, 100 mm, or 150 mm dishes; multi-well plates (e.g., 6-well plates, 12-well plates, 24-well plates, 48-well plates, or 96 well plates); flasks (e.g., T-flasks, e.g., T-25 flasks, T-75 flasks, or T-160 flasks); or shaker flasks.

In some embodiments, host cells described herein are cultured in a bioreactor. In some embodiments, a bioreactor is used for culturing adherent cells. In other embodiments, a bioreactor is used for culturing suspension cells. In some embodiments, a bioreactor comprises or is a continuous flow batch bioreactor. In some embodiments, a bioreactor comprises or is a perfusion bioreactor. In some embodiments, a bioreactor comprises or is a batch process bioreactor. In some embodiments, a bioreactor comprises or is a fed batch bioreactor. An exemplary bioreactor is a fixed bed bioreactor, e.g., an iCELLis bioreactor. A bioreactor can be maintained under conditions sufficient to produce recombinant viral vector particles (e.g., rAAV particles). Culture conditions can be modulated to optimize yield, purity, and/or structure of recombinant viral vector particles (e.g., rAAV particles).

In some embodiments, a plurality of recombinant viral vector particles (e.g., rAAV particles) are produced in a large-scale preparation comprising host cells (e.g., suspension or adherent host cells). In some embodiments, a large-scale preparation of a plurality of recombinant viral vector particles (e.g., rAAV particles) is produced in a bioreactor. In some embodiments, a bioreactor comprises at least about 1 liter of culture media, e.g., between about 1 liter to about 2,000 liters, about 1,000 liters to about 2,000 liters, about 500 liters to about 1,500 liters, about 250 liters to about 1,250 liters, about 1,500 liters to about 2,000 liters, about 1,000 liters to about 1,500 liters, about 500 liters to about 1,750 liters, about 250 liters to about 1,500 liters, about 1,500 liters to about 2,000 liters, about 10 liters to about 800 liters, about 25 liters to about 500 liters, about 2 liters to about 300 liters, about 100 liters to about 400 liters, about 50 liters to about 600 liters, about 15 liters to about 800 liters, about 80 liters to about 500 liters, about 20 liters to about 300 liters, about 100 liters to about 400 liters, or about 50 liters to about 600 liters of culture media, e.g., at least about 2 liters, about 3 liters, about 4 liters, about 5 liters, about 10 liters, about 15 liters, about 20 liters, about 25 liters, about 30 liters, about 35 liters, about 40 liters, about 45 liters, about 50 liters, about 55 liters, about 60 liters, about 65 liters, about 70 liters, about 75 liters, about 80 liters, about 85 liters, about 90 liters, about 100 liters, about 125 liters, about 150 liters, about 175 liters, about 200 liters, about 225 liters, about 250 liters, about 275 liters, about 300 liters, about 350 liters, about 400 liters, about 450 liters, about 500 liters, about 550 liters, about 575 liters, 600 liters, about 625 liters, about 650 liters, about 675 liters, about 700 liters, about 725 liters, about 750 liters, about 775 liters, about 800 liters, about 850 liters, about 900 liters, about 950 liters, or about 1,000 liters of culture media.

In some embodiments, a bioreactor is maintained under conditions that promote growth of a host cell described herein, e.g., at a temperature (e.g., about 37° C.) and gas concentration (e.g., 5% to 10% CO₂) that is permissive for host cell growth. For example, a bioreactor can perform one or more of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), cleaning, and/or sterilization.

Exemplary bioreactor units, may contain multiple reactors within a unit, e.g., a unit can comprise 1, 2, 3, 4, 5, 10, 15, 20, 25, 30, 35, 40, 45, 50, 60, 70, 80, 90, 100, or more bioreactors. Any suitable bioreactor diameter and/or shape can be used. In some embodiments, suitable reactors can be round, e.g., cylindrical. In some embodiments, suitable reactors can be square, e.g., rectangular.

The foregoing methods for producing recombinant viral vector particles (e.g., rAAV particles) are not meant to be limiting, and other suitable methods will be apparent to the skilled artisan.

Recombinant Viral Vectors

The present disclosure, among other things, provides methods, compositions, and systems for producing recombinant viral vector particles (e.g., adeno-associated viral (AAV) particles). Recombinant viral vectors have become widely used for inserting genes into mammalian cells (e.g., human cells). Many forms of viral vectors can be used to deliver a payload (e.g., a payload described herein) to a cell, tissue, or organism.

Non-limiting examples of recombinant viral vectors include, but are not limited to, adeno-associated virus (AAV), retrovirus (e.g., Moloney murine leukemia virus (MMLV), Harvey murine sarcoma virus, murine mammary tumor virus, or Rous sarcoma virus), adenovirus, SV40-type virus, polyomavirus, Epstein-Barr virus, papilloma virus, herpes virus, vaccinia virus, or polio virus.

In some embodiments, a recombinant viral vector comprises or is a retroviral vector. Retroviruses are enveloped viruses that belong to viral family Retroviridae. Protocols for production of replication-deficient retroviruses are known in the art (See, e.g., Kriegler, M., Gene Transfer and Expression, A Laboratory Manual, W. H. Freeman Co., New York (1990) and Murry, E. J., Methods in Molecular Biology, Vol. 7, Humana Press, Inc., Cliffton, N.J. (1991), each of which is hereby incorporated by reference in its entirety). A number of retroviral systems are known in the art (See, e.g., U.S. Pat. Nos. 5,994,136, 6,165,782, and 6,428,953, each of which is hereby incorporated by reference in its entirety). In some embodiments, a retrovirus comprises or is a lentivirus of Retroviridae family. In some embodiments, a lentivirus comprises or is human immunodeficiency viruses (e.g., HIV-1 or HIV-2), simian immunodeficiency virus (S1V), feline immunodeficiency virus (FIV), equine infections anemia (EIA), or visna virus.

In some embodiments, a recombinant viral vector comprises or is an adenovirus vector. An adenovirus vector may be from any origin, subgroup, subtype, serotype, or mixture thereof. For instance, an adenovirus can be of subgroup A (e.g., serotypes 12, 18, or 31), subgroup B (e.g., serotypes 3, 7, 11, 14, 16, 21, 34, 35, or 50), subgroup C (e.g., serotypes 1, 2, 5, or 6), subgroup D (e.g., serotypes 8, 9, 10, 13, 15, 17, 19, 20, 22-30, 32, 33, 36-39, or 42-48), subgroup E (e.g., serotype 4), subgroup F (e.g., serotypes 40 or 41), an unclassified serogroup (e.g., serotypes 49 or 51), or any other adenoviral serotype. Adenoviral serotypes 1 through 51 are available from the American Type Culture Collection (ATCC, Manassas, Va.).

Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087, each of which is hereby incorporated by reference in its entirety. Further examples of adenoviral vectors can be found in U.S. Publication Nos. 20150093831, 20140248305, 20120283318, 20100008889, 20090175897 and 20090088398, each of which is hereby incorporated by reference in its entirety.

In some embodiments, a recombinant viral vector comprises or is an alphavirus. Exemplary alphaviruses include, but are not limited to, Sindbis virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, a genome of such viruses encodes nonstructural (e.g., replicon) and structural proteins (e.g., capsid and envelope) that can be translated in host cell cytoplasm. Ross River virus, Sindbis virus, Semliki Forest virus (SFV), and Venezuelan equine encephalitis virus (VEEV) have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819, each of which is incorporated herein by reference in their entirety

In some embodiments, a recombinant viral vector comprises or is an AAV vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is hereby incorporated by reference in its entirety). Methods for generating and using AAV vectors are described, for example, in U.S. Pat. Nos. 5,139,941 and 4,797,368, each of which is hereby incorporated by reference in its entirety.

Generally, AAV vectors for use in methods, compositions, and systems described herein may be of any AAV serotype. AAV serotypes generally have different tropisms to infect different tissues. In some embodiments, an AAV serotype is selected based on a tropism. Several AAV serotypes have been characterized including, but not limited to, AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, as well as variants or hybrids thereof. For example, in some embodiments, an AAV vector comprises or is an AAV2/5, AAV2/6, AAV2/8 or AAV2/9 vector (e.g., AAV6, AAV8 or AAV9 serotype having AAV2 ITR).

In some embodiments, an AAV vector is derived from an AAV genome sequence or a variant thereof as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

In some embodiments, an AAV serotype may have or comprise a mutation in an AAV9 sequence (e.g., as described in N Pulicherla et al. Molecular Therapy 19(6): 1070-1078 (2011), which is hereby incorporated by reference in its entirety). AAV9 serotypes may include, but not limited to, AAV9.68, AAV9.9, AAV9.11, AAV9.13, AAV9.16, AAV9.24, AAV9.45, AAV9.47, AAV9.61, and AAV9.84. In certain embodiments, an AAV9 variant comprises or is AAVhu68 or a variant thereof (e.g., as described in WO 2018/160585, which is hereby incorporated by reference in its entirety). Other AAV vectors are described in, e.g., Sharma et al., Brain Res Bull. 2010 Feb. 15; 81 (2-3): 273, which is hereby incorporated by reference in its entirety.

In some embodiments, an AAV vector comprises or is a naturally occurring AAV. In some embodiments, an AAV vector is a modified AAV or a variant of a naturally occurring AAV. In some embodiments, an AAV vector may be generated by directed evolution, e.g., by DNA shuffling, peptide insertion, or random mutagenesis, in order to introduce modifications into the AAV sequence to improve one or more properties for gene therapy. In some embodiments, such modifications avoid or lessen an immune response or recognition by neutralizing antibodies and/or allow for more efficient and/or targeted transduction (See, e.g., Asuri et al., Molecular Therapy 20.2 (2012): 329-338, which is hereby incorporated by reference in its entirety). Methods of using directed evolution to engineer an AAV vector can be found, e.g., in U.S. Pat. No. 8,632,764, which is hereby incorporated by reference in its entirety. In some embodiments, a modified AAV is modified to include a specific tropism.

In some embodiments, an AAV vector may be a dual or triple AAV vector, e.g., for the delivery of large payloads (e.g., payloads of greater than approximately 5 kb) and/or to address safety concerns associated with administration of single AAV vectors. In some embodiments, a dual AAV vector may include two separate AAV vectors, each including a fragment of a full sequence of a large payload of interest, and when recombined, the fragments form the full sequence of the large payload of interest or a functional portion thereof. In some embodiments, a triple AAV vector may include three separate AAV vectors, each including a fragment of a sequence of a large payload of interest, and when recombined, the fragments form the full sequence of the large payload of interest or a functional portion thereof.

Multiple AAV (e.g., dual or triple AAV vectors) can be delivered to and co-transduced into the same cell, where fragments of a payload of interest recombine and generate a single mRNA transcript of the entire payload of interest. In some embodiments, fragmented payloads include a non-overlapping sequences. In some embodiments, fragmented payloads include a specified overlapping sequences. In some embodiments, multiple AAV vectors for dual or triple transfection may be the same type of AAV vector (e.g., same serotype and/or same construct). In some embodiments, multiple AAV vectors of dual or triple may transfection be different types of AAV vector (e.g., different serotype or different construct).

In some embodiments, an AAV vector comprises a single-stranded (ss) or self-complementary (sc) AAV nucleic acid vector. In some embodiments, an AAV vector comprises an expression construct and one or more regions comprising ITR sequences (e.g., wild-type ITR sequences or engineered ITR sequences) flanking an expression construct. In some embodiments, an AAV vector is encapsidated by a viral capsid. In some embodiments, a viral capsid comprises 60 capsid protein subunits. In some embodiments, a viral capsid comprises VP1, VP2, and VP3. In some embodiments, VP1, VP2, and VP3 subunits are present in a capsid at a ratio of about 1:1:10, respectively.

ITR sequences of an AAV vector can be derived from any AAV serotype (e.g., AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, or variants or hybrids thereof). In some embodiments, ITR sequences are derived from one or more other serotypes, e.g., as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

ITR sequences and plasmids containing ITR sequences are known in the art and are commercially available (See, e.g., products and services available from Vector Biolabs, Philadelphia, PA; Cellbiolabs, San Diego, CA; Agilent Technologies, Santa Clara, Ca; and Addgene, Cambridge, MA; and described in Kessler et al. PNAS. 1996 Nov. 26; 93(24): 14082-7; Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus; and U.S. Pat. Nos. 5,139,941 and 5,962,313; each of which is hereby incorporated by reference in its entirety).

An AAV vector may comprise or be based on a serotype selected from any following serotypes or variants thereof including, but not limited to, AAV9.68, AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/rh.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV5, AAV-8b, AAV-8h, AAV9, AAV9.11, AAV9.13, AAV9.16, AAV9.24, AAV9.45, AAV9.47, AAV9.61, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and AAV SM 10-8.

An AAV serotype may be from any number of species. For example, an AAV may be or comprise an avian AAV (AAAV), e.g., as described in U.S. Pat. No. 9,238,800, which is hereby incorporated by reference in its entirety. An AAV serotype may be or comprise a bovine AAV (BAAV), e.g., as described in U.S. Pat. No. 9,193,769 or 7,427,396, each of which is hereby incorporated by reference in its entirety. An AAV may be or comprise a caprine AAV, e.g., as described in U.S. Pat. No. 7,427,396, which is hereby incorporated by reference in its entirety. An AAV serotype may also be a variant or hybrid of any of the foregoing.

In some embodiments, an AAV may be or comprise a serotype generated from an AAV9 capsid library with mutations in amino acids 390 to 627 (VP1 numbering), e.g., as described in Pulicherla et al. (Molecular Therapy 19(6): 1070-1078 (2011), which is hereby incorporated by reference in its entirety. An AAV serotype (with corresponding nucleotide and amino acid substitutions) may include, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F4111), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (CI 531 A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A,G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L), and AAV9.95 (T1605A; F535L).

In some embodiments, an AAV vector comprises a capsid including modified capsid proteins (e.g., capsid proteins comprising a modified VP3 region). Methods of producing modified capsid proteins are known in the art (See, e.g., US20130310443, which is hereby incorporated by reference in its entirety). In some embodiments, an AAV vector comprises a modified capsid protein comprising at least one non-native amino acid substitution at a position that corresponds to a surface-exposed amino acid (e.g., a surface exposed tyrosine) in a wild-type capsid protein. In some embodiments, an AAV vector comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in a wild-type capsid protein, a non-lysine amino acid (e.g., a glutamic acid) at a position that corresponds to a surface-exposed lysine amino acid in a wild-type capsid protein, a non-serine amino acid (e.g., a valine) at a position that corresponds to a surface-exposed serine amino acid in a wild-type capsid protein, or a combination thereof. In some embodiments, an AAV vector comprises a capsid that includes modified capsid proteins having at least 1, 2, 3, 4, 5, 6, 7, 8, 9, 10, or more amino acid substitutions.

Additional methods for generating and isolating AAV viral vectors suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which are hereby incorporated by reference in their entirety.

Compositions

The present disclosure, among other things, provides a composition comprising a plurality of recombinant viral vector particles (e.g., rAAV particles) formed by methods described herein. In some embodiments, a composition comprises a pharmaceutical composition comprising at least one pharmaceutically acceptable component (e.g., a pharmaceutically acceptable carrier, diluent, or excipient). Such pharmaceutical compositions are useful for, among other things, administration to a subject in vivo or ex vivo.

The term “pharmaceutical composition,” as used herein, refers to a composition comprising rAAV particles that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to achieve a desired therapeutic effect when administered to a subject or population in need thereof (e.g., by a statistically significant probability). A pharmaceutical composition may be specially formulated for administration in solid or liquid form. In some embodiments, a pharmaceutical composition is formulated for administration by parenteral administration, such as by subcutaneous, intramuscular, intravenous or epidural injection. In some embodiments, a pharmaceutical composition is formulated as a sterile solution or suspension, e.g., in a sustained-release formulation. Pharmaceutical compositions of the disclosure may be formulated for administration by injection or infusion (e.g., subcutaneous, intramuscular, intravenous or epidural injection or infusion). For example, compositions may be formulated for administration by retinal, subretinal, intravitreal, suprachoroidal, intraspinal, intracisterna magna, or intrathecal injection or infusion. In some embodiments, a pharmaceutical composition is intended and suitable for administration to a human subject. In some embodiments, a pharmaceutical composition is substantially free of contaminants (e.g., sterile and substantially pyrogen-free). Formulations of the pharmaceutical compositions may include, but are not limited to, formulations for oral administration, such as drenches (aqueous or non-aqueous solutions or suspensions), tablets (e.g., targeted for buccal, sublingual, and systemic absorption), boluses, powders, granules, pastes for application to the tongue; topical application, such as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

In some embodiments, pharmaceutical compositions also contain a pharmaceutically acceptable carrier, excipient, or diluent. Such excipients include any pharmaceutical agent, e.g., a pharmaceutical agent that does not itself induce an immune response harmful to the individual receiving the composition, and which may be administered without undue toxicity. Pharmaceutically acceptable excipients include, but are not limited to, liquids, such as water, saline, glycerol, sugars, and ethanol. Pharmaceutically acceptable salts can also be included therein, for example, mineral acid salts, such as hydrochlorides, hydrobromides, phosphates, or sulfates; and the salts of organic acids, such as acetates, propionates, malonates, or benzoates. Additionally, auxiliary substances, such as wetting or emulsifying agents or pH buffering substances, may be present in such vehicles.

Pharmaceutical compositions may be provided as a salt and can be formed with many acids, including but not limited to, hydrochloric, sulfuric, acetic, lactic, tartaric, malic, or succinic. Salts tend to be more soluble in aqueous or other protonic solvents than corresponding free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, and isotonic and absorption promoting or delaying agents, compatible with pharmaceutical administration or in vivo contact or delivery. Aqueous and non-aqueous solvents, solutions, and suspensions may include suspending agents and thickening agents. Such pharmaceutically acceptable carriers include tablets (coated or uncoated), capsules (hard or soft), microbeads, powder, granules, and crystals. Supplementary active compounds (e.g., preservatives, antibacterial, antiviral, and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable, or synthetic oils. Aqueous injection suspensions may contain substances that increase the viscosity of a suspension, such as sodium carboxymethyl cellulose, sorbitol, or dextran. Additionally, suspensions may be prepared as appropriate oil injection suspensions. Suitable lipophilic solvents or vehicles include fatty oils, such as sesame oil, or synthetic fatty acid esters, such as ethyl oleate or triglycerides, or liposomes. Optionally, suspension may also contain suitable stabilizers or agents that increase solubility to allow for preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

Administration

The present disclosure, among other things, provides methods of administering a composition (e.g., a pharmaceutical composition) comprising a plurality of rAAV particles formed by methods described herein. Compositions (e.g., pharmaceutical compositions) comprising recombinant viral vector particles (e.g., rAAV particles) produced with the methods described herein can be used to treat any disease or disorder, e.g., subjects suffering from or susceptible to a disease or disorder described herein. The route and/or mode of administration can vary depending upon the desired results. One with skill in the art (e.g., a physician), is aware that dosage regimens can be adjusted to provide the desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. Mode of administration is left to discretion of a practitioner.

As used herein, the term “administration” refers to the administration of a composition comprising recombinant viral vector particles (e.g., rAAV particles) as described herein to a subject. Administration may be by any appropriate route. For example, in some embodiments, administration may be local or systemic administration (e.g., to a mammal, e.g., to a human, e.g., a patient). A composition of the disclosure may be administered by injection or infusion by any route. For example, a composition may be administered by retinal, subretinal, intravitreal, suprachoroidal, intraspinal, intracisterna magna, or intrathecal injection or infusion. Additional exemplary routes of administration may include, but are not limited to, bronchial (e.g., bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (e.g., intratracheal instillation), transdermal, vaginal, and vitreal.

For example, a composition may be administered by retinal, subretinal, intravitreal, or suprachoroidal injection or infusion. Additional exemplary routes of administration may include, but are not limited to, bronchial (e.g., bronchial instillation), buccal, enteral, interdermal, intra-arterial, intracisterna magna (ICM), intradermal, intragastric, intramedullary, intramuscular, intranasal, intra-parenchymal (e.g., intra-thalamic), intraperitoneal, intrathecal, intravenous, intraventricular, mucosal, nasal, oral, rectal, intraspinal, spinal sub-pial, subcutaneous, sublingual, topical, tracheal (e.g., intratracheal instillation), transdermal, vaginal, and vitreal administration.

Methods and uses disclosed herein include delivery and administration systemically, regionally or locally, or by any route, for example, by injection or infusion. A composition (e.g., pharmaceutical composition) comprising a plurality of recombinant viral vector particles (e.g., rAAV particles) formed by methods described herein may be administered by injection or infusion by any route. For example, a composition may be administered by retinal, subretinal, intravitreal, suprachoroidal, intraspinal, intracisterna magna, or intrathecal injection or infusion.

Delivery of a pharmaceutical composition in vivo may generally be accomplished via injection using a conventional syringe, although other delivery methods such as convection-enhanced delivery can also be used. For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in treatment of patients with certain diseases or disorders may determine the optimal route for administration of vectors described herein.

The disclosure provides methods for introducing a composition (e.g., a pharmaceutical composition) comprising recombinant viral vector particles (e.g., rAAV particles) described herein into a cell, a tissue, or an animal. In some embodiments, such methods comprise contacting a cell, a tissue, or an animal with a composition comprising rAAV particles described herein, such that at least one payload is expressed or present in the cell, tissue, or animal.

The disclosure also provides methods for administering a composition (e.g., a pharmaceutical composition) comprising recombinant viral vector particles (e.g., rAAV particles) described herein to a subject. In some embodiments, such methods include administering to a subject (e.g., a mammal), a composition comprising recombinant viral vector particles (e.g., rAAV particles) described herein, such that at least one payload is expressed or present in the subject (e.g., in a cell or tissue of a subject). In some embodiments, a method includes providing cells of a subject (e.g., a mammal) with a composition (e.g., a pharmaceutical composition) comprising recombinant viral vector particles (e.g., rAAV particles) described herein, such that at least one payload is expressed or present in the subject.

A composition (e.g., a pharmaceutical composition) comprising recombinant viral vector particles (e.g., rAAV particles) described herein can be administered in a sufficient or effective amount to a subject in need thereof. Doses can vary and depend upon a type, onset, progression, severity, frequency, duration, or probability of disease to which treatment is directed, the clinical endpoint desired, previous or simultaneous treatments, the general health, age, gender, race or immunological competency of the subject, and other factors that will be appreciated by a skilled artisan. Dose amount, number, frequency, or duration may be proportionally increased or reduced, as indicated by any adverse side effects, complications, or other risk factors of treatment and status of the subject. A skilled artisan will appreciate the factors that may influence the dosage and timing required to provide an amount sufficient for providing a therapeutic or prophylactic benefit.

A dose to achieve a therapeutic effect will vary based on several factors including, but not limited to: route of administration, level of payload or payload expression required to achieve a therapeutic effect, specific disease treated, any host immune response, and stability of payload or payload expression. One skilled in the art can determine a dose range to treat a patient having a particular disease or disorder based on the aforementioned factors, as well as other factors.

An effective amount or a sufficient amount can (but need not) be provided in a single administration, may require multiple administrations, and, can (but need not) be, administered alone or in combination with another composition. For example, an amount may be proportionally increased as indicated by need of a subject, type, status, and severity of disease treated or side effects (if any) of treatment. Amounts considered effective also include amounts that result in a reduction of use of another treatment, therapeutic regimen, or protocol.

Accordingly, pharmaceutical compositions include compositions comprising rAAV particles in an effective amount to achieve an intended therapeutic purpose. Determining a therapeutically effective dose is well within the capability of a skilled medical practitioner using techniques and guidance provided herein. Therapeutic doses can depend on, among other factors, age and general condition of a subject, severity of a disease or disorder, and payload amount or expression in a subject. Thus, a therapeutically effective amount in humans will fall in a relatively broad range that may be determined by a medical practitioner based on response of an individual patient to rAAV-based treatment. Pharmaceutical compositions may be delivered to a subject so as to allow production of a payload described herein in vivo by gene- and or cell-based therapies or by ex-vivo modification of a patient's or donor's cells.

In some embodiments, a composition (e.g., a pharmaceutical composition) comprising recombinant viral vector particles (e.g., rAAV particles) described herein may be administered to a subject once daily, weekly, every 2, 3, or 4 weeks, or even at longer intervals. In some embodiments, a composition (e.g., a pharmaceutical composition) comprising recombinant viral vector particles (e.g., rAAV particles) described herein may be administered according to a dosing regimen that includes (i) an initial administration that is once daily, weekly, every 2, 3, or 4 weeks, or even at longer intervals; followed by (ii) a period of no administration of, e.g., 1, 2, 3, 4, 5, 6, 8, or 10 months, or 1, 2, 3, 4, 5, 6, 7, 8, 9, or 10 years. In some embodiments, a composition (e.g., a pharmaceutical composition) comprising recombinant viral vector particles (e.g., rAAV particles) described herein may be administered (i) one or more times during an initial time period of up to 2, 4, or 6 weeks or less; followed by (ii) a period of no administration of, e.g., 1, 2, 3, 4, 5, 6, 7, 8, 9, or 10 years. In some embodiments, a subject is monitored before and/or following treatment with a composition (e.g., a pharmaceutical composition) comprising recombinant viral vector particles (e.g., rAAV particles) described herein.

Uses

The present disclosure, among other things, provides methods of delivering a gene therapy as described herein to a cell or tissue.

The present disclosure also provides methods of treating a subject with a composition (e.g., a pharmaceutical composition) comprising a plurality of recombinant viral vector particles (e.g., rAAV particles) as described herein. The term “subject,” as used herein, refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from a disease, disorder or condition, e.g., a disease, disorder or condition that can be treated as provided herein, e.g., a neurological disease or disorder or a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., a clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

In some embodiments, methods and kits of the present invention may be used for the evaluation and/or monitoring of gene therapy. The term “gene therapy,” as used herein, refers to insertion or deletion of specific genomic DNA sequences to treat or prevent a disorder or condition for which such therapy is sought. In some embodiments, the insertion or deletion of genomic DNA sequences occurs in specific cells (e.g., target cells). Target cells may be from a mammal and/or may be cells in a mammalian subject. Mammals include, but are not limited to, humans, dogs, cats, cows, sheep, pigs, or llamas. In some embodiments, heterologous DNA is transferred to target cells. The heterologous DNA may be introduced into the selected target cells in a manner such that the heterologous DNA is expressed and a therapeutic product encoded thereby is produced. Additionally or alternatively, the heterologous DNA may in some manner mediate expression of DNA that encodes the therapeutic product, or it may encode a product, such as a peptide or RNA that in some manner mediates or modulates, directly or indirectly, expression of a therapeutic product. Genetic therapy may also be used to deliver nucleic acid encoding a gene product that replaces a defective gene or supplements a gene product produced by the mammal or the cell in which it is introduced. The heterologous DNA encoding the therapeutic product may be modified prior to introduction into the cells of the afflicted host in order to enhance or otherwise alter the product or expression thereof. Genetic therapy may also involve delivery of an inhibitor or repressor or other modulator of gene expression. Such an inhibitor or repressor or other modulator can be a polypeptide, peptide, or nucleic acid (e.g., DNA or RNA). Gene therapy may include in vivo or ex vivo techniques. In some embodiments, viral and non-viral based gene transfer methods can be used to introduce a nucleic acid encoding a polypeptide of interest or to introduce a therapeutic nucleic acid into mammalian cells or target tissues. Non-viral vector delivery systems include DNA plasmids, naked nucleic acid, and nucleic acid complexed with a delivery vehicle, such as poloxamers or liposomes. Viral vector delivery systems include DNA and RNA viruses, which have either episomal or integrated genomes after delivery to the cell.

In some embodiments, gene therapy comprises administration of a composition (e.g., a pharmaceutical composition) comprising a plurality of recombinant viral vector particles (e.g., rAAV particles) described herein. In some embodiments, samples for evaluating and/or monitoring gene therapy may be obtained prior to the initiation of gene therapy. In some embodiments, samples are obtained after a first gene therapy treatment or dose. In some embodiments, samples are obtained after the conclusion of gene therapy. In some embodiments, samples are obtained at specific time points, intervals, or any other metric of time before, during, or after gene therapy is performed.

In some embodiments, a composition (e.g., a pharmaceutical composition) comprising a plurality of recombinant viral vector particles (e.g., rAAV particles) described herein is administered to a subject suffering from or at risk of a disease, disorder, or condition. In some embodiments, a composition (e.g., a pharmaceutical composition) comprising a plurality of recombinant viral vector particles (e.g., rAAV particles) described herein is administered in combination with one or more additional therapeutics agents to a subject. In some embodiments, a composition (e.g., a pharmaceutical composition) comprising a plurality of recombinant viral vector particles (e.g., rAAV particles) described herein is contacted with an organ, tissue, or cells ex vivo. The organ, tissue, or cells can be introduced into a subject and can be protected from damage that would otherwise be caused by the recipient's immune system.

### INCORPORATION BY REFERENCE

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLE

The following Example is put forth so as to provide those of ordinary skill in the art with a complete disclosure and description of how to make and use the present invention, and is not intended to limit the scope of what the inventors regard as their invention nor are they intended to represent that the experiments below are all or the only experiments performed.

### Example 1: Production of rAAV Using Microtubule Destabilizing Agents

The present Example demonstrates the effect of microtubule destabilizing agents on titer (e.g., genome titer) of recombinant adeno-associated virus (rAAV) particles by transient transfection of HEK293 cells.

A variety of cell cycle arresting agents (aphidicolin, caffeine, colcemid, colchicine, flavopiridol, hydroxyurea, thymidine, trichostatin A, and vinblastine sulfate) were screened for effects on production of rAAV particles. Cell cycle arresting agents were obtained from Santa Cruz Biotechnology (SCBT) or Sigma. Aphidicolin, colcemid, flavopiridol, and trichostatin A were dissolved in dimethyl sulfoxide (DMSO) then diluted in cell culture medium. Caffeine, colchicine, hydroxyurea, thymidine, and vinblastine sulfate were directly dissolved in cell culture medium to create working stocks.

Addition of cell cycle arresting agents to rAAV production was evaluated using transient transfection of suspension HEK293 host cells with plasmids encoding Payload C flanked by an ITR on either side, at least one Rep polypeptide, at least one Cap polypeptide, and at least one helper polypeptide. HEK293 host cells were seeded at about 5.0×10⁵ vc/ml to about 5.0×10⁶ vc/ml, and grown in cell culture media for 4 to 96 hours. Complexing of polyethylenimine (PEI) transfection reagent and plasmid DNA was then performed for 5 to 120 minutes. Transfection complexes were then diluted and added to cell culture.

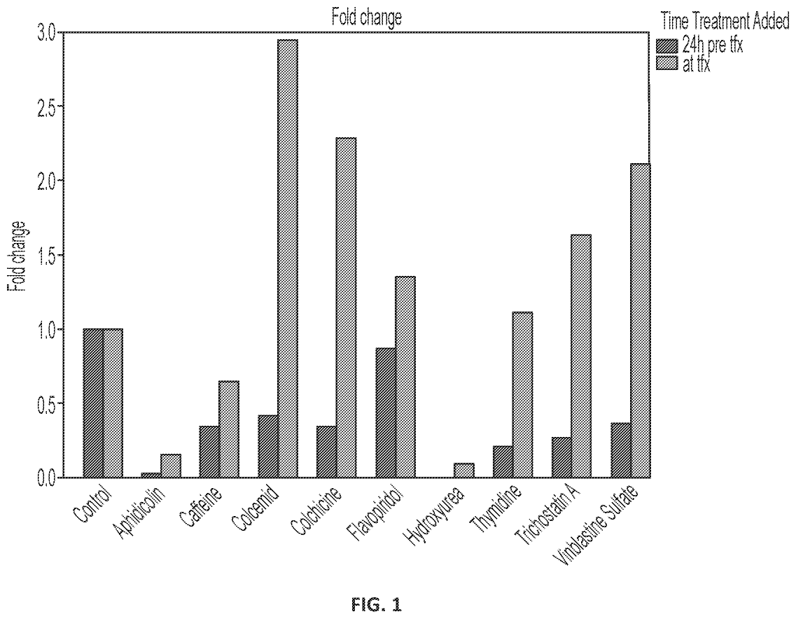

Addition of cell cycle arresting agents (aphidicolin, caffeine, colcemid, colchicine, flavopiridol, hydroxyurea, thymidine, trichostatin A, and vinblastine sulfate) at 24 hours prior to transfection (24 h pre tfx) and at 0.5 hours after transfection (at tfx) was evaluated in shake flask. Addition at 0.5 hours post-transfection of cell cycle arresting agents was chosen as the time of addition for all experiments based on the fold-change in AAV titer achieved (FIG. 1). Use of microtubule destabilizing agents (colcemid, colchicine, and vinblastine sulfate) resulted in improvements in titer of about 2-fold higher titer to about 3-fold higher tier relative to control titers at 0.5 hours post-transfection.

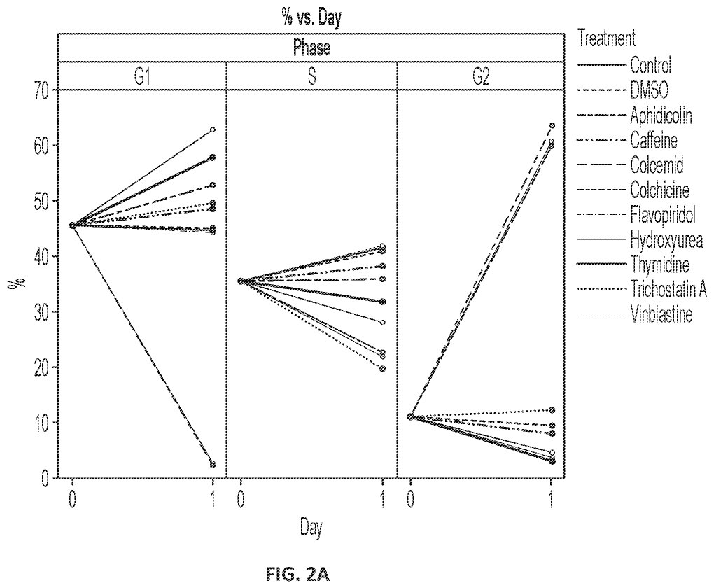

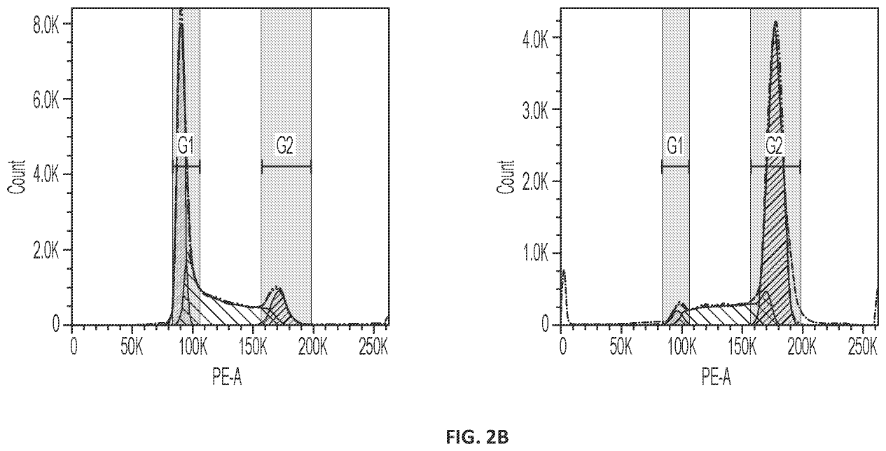

Cell cycle phase of non-transfected, suspension HEK293 cultures was evaluated using fluorescence activated cell sorting (FACS). Suspension HEK293 cultures were grown in shake flasks for 24 hours with cycle arresting agents (aphidicolin, caffeine, colcemid, colchicine, flavopiridol, hydroxyurea, thymidine, trichostatin A, and vinblastine sulfate) prior to sampling for FACS analysis using propidium iodide (PI), which binds to DNA. Light intensity is indicative of the relative quantity of DNA present in a cell, which is a measure of cell phase. Forward scatter indicates the a size of a cell, while side scatter indicates the a size of intracellular components. Fluorescence of PI was measured using an excitation of 493 nm and emission of 646 nm. Use of microtubule destabilizing agents (colcemid, colchicine, and vinblastine sulfate) resulted in cell synchronization in non-transfected, suspension HEK293 cell cultures in G2 phase (FIG. 2A). Cell cycle arrest of suspension HEK293 cell cultures by colcemid in G2/M phase is shown in FIG. 2B.

The effect of different concentrations of microtubule destabilizing agents on fold changes in rAAV particle titers was determined. Colcemid, colchicine, and vinblastine sulfate were screened in addition to vincristine sulfate, which was included in the screen based on a similar mechanism of action as vinblastine sulfate. Concentrations of each microtubule destabilizing agent screened in shake flasks) at 0.5 hours post-transfection are shown in Table 1.

TABLE 1
Concentrations of microtubule destabilizing agents screened.

| | Concentrations Screened (uM) | | | |
|---|---|---|---|---|
| Compound | Control | 1× | 2× | 5× |
| Colcemid | 0 | 0.2 | 0.4 | 1 |
| Colchicine | 0 | 2.5 | 5 | 12.5 |
| Vinblastine Sulfate | 0 | 0.5 | 1 | 2.5 |
| Vincristine Sulfate | 0 | 0.5 | 1 | 2.5 |

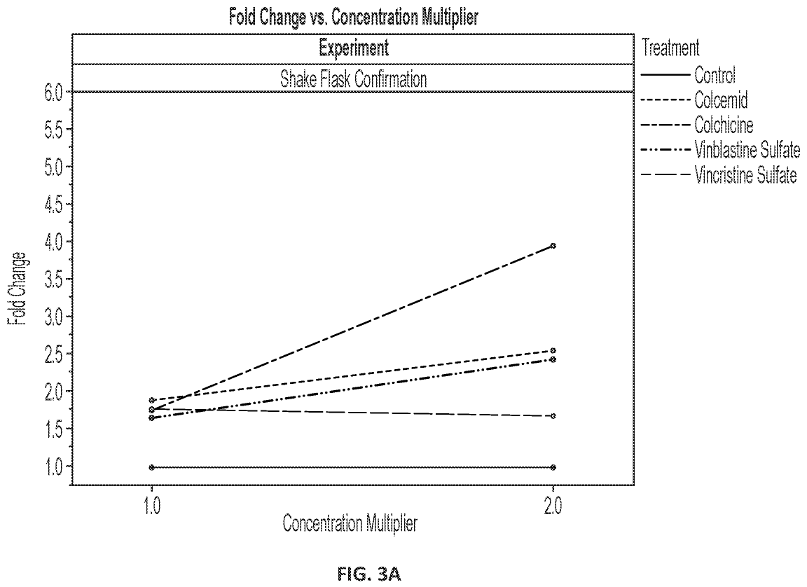

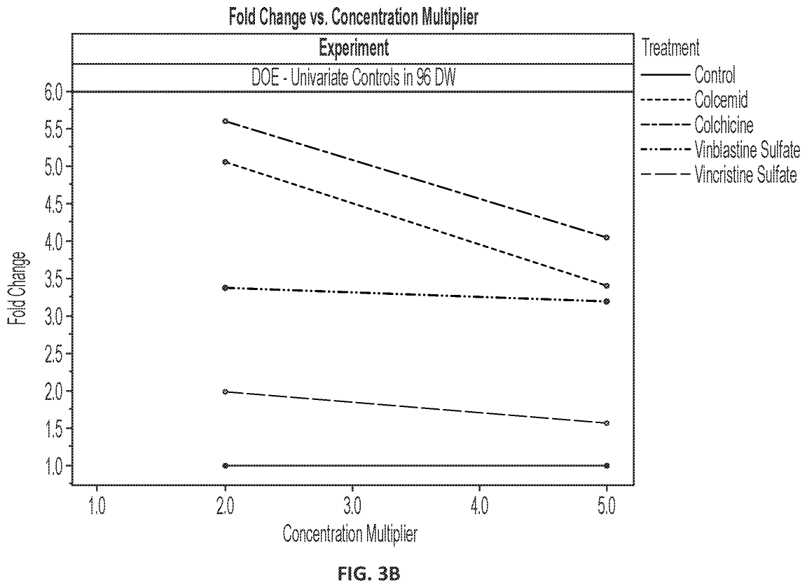

Fold changes in rAAV particle titer following transient transfection of suspension HEK293 cells with plasmids encoding Payload C flanked by an ITR on either side, at least one Rep polypeptide, at least one Cap polypeptide, and at least one helper polypeptide as well as addition of microtubule destabilizing agents (colcemid, colchicine, vinblastine sulfate, and vincristine sulfate) were compared at 1× and 2× concentrations (FIG. 3A) and 2× and 5× concentrations (FIG. 3B) at 0.5 hours post-transfection. Concentrations of 2× for microtubule destabilizing agents were most effective.

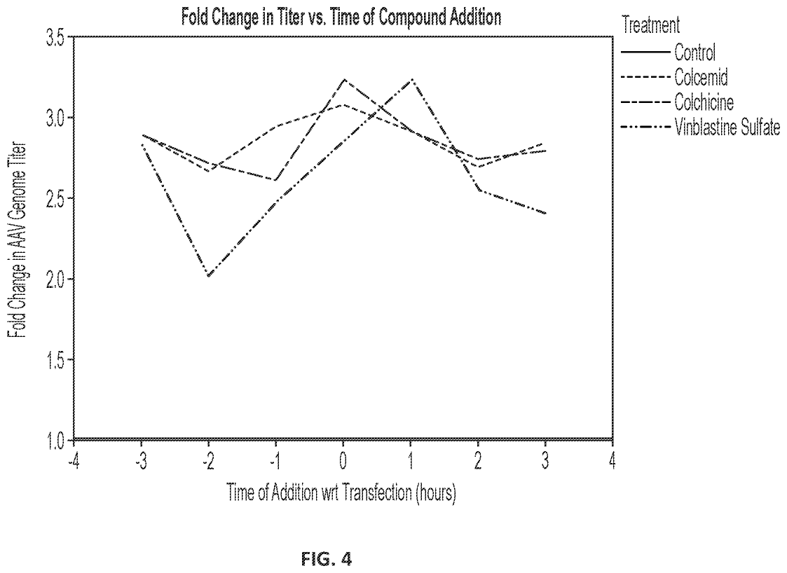

The effect of time of addition of microtubule destabilizing agents on fold change in rAAV particle titer was also determined. Microtubule destabilizing agents (colcemid, colchicine, and vinblastine sulfate) were added at different time points (3 hours, 2 hours, and 1 hour pre-transfection and 0.5 hour, 2 hour, and 3 hours post-transfection) at 2.5× concentrations around transient transfection of suspension HEK293 cells with plasmids encoding Payload C flanked by an ITR on either side, at least one Rep polypeptide, at least one Cap polypeptide, and at least one helper polypeptide in shake flasks. Addition at 0.5 hour post-transfection was selected for future experiments based on increases in fold changes in rAAV particle titer of about 3-fold or greater (FIG. 4).

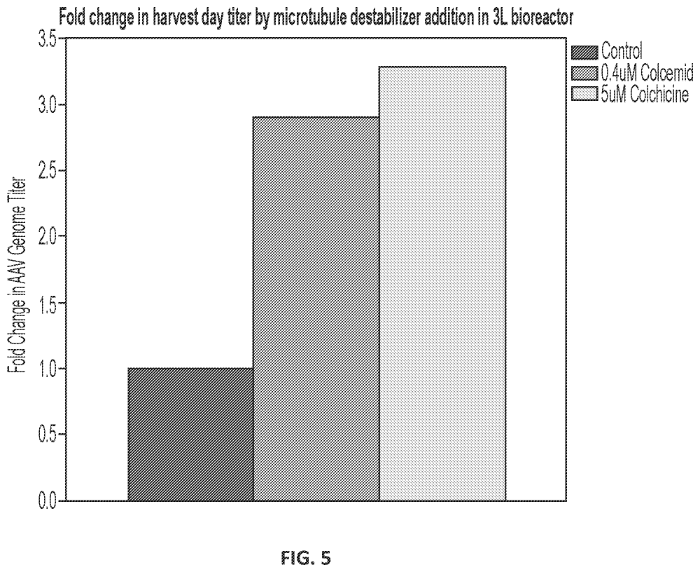

Scalability of the use of microtubule destabilizing agents (colcemid and colchicine) to increase rAAV particle titer was assessed in bioreactors. Suspension HEK293 cells were transiently transfected with plasmids encoding Payload C flanked by an ITR on either side, at least one Rep polypeptide, at least one Cap polypeptide, and at least one helper polypeptide, microtubule destabilizing agents colcemid (2× concentration of 0.4 μM) or colchicine (2× concentration of 5 μM) were added at 0.5 hours post-transfection, and HEK293 cells were cultured in 3-liter bioreactors for seven days prior to sampling. Fold changes in rAAV particle titer of about 3-fold or greater were obtained for both colcemid and colchicine in bioreactors (FIG. 5).

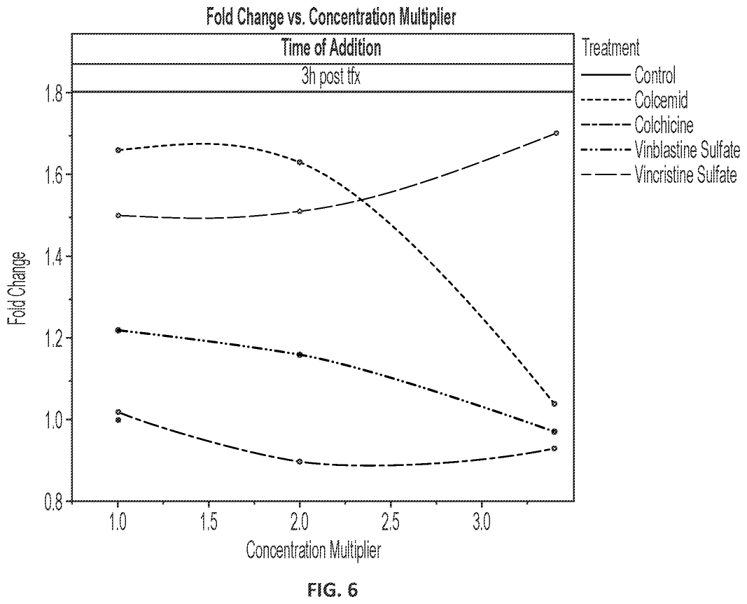

The effect of microtubule destabilizing agents (colcemid, colchicine, vinblastine sulfate, and vincristine sulfate) on fold changes in rAAV particle titer of adherent HEK293 cells in T flasks was also determined. HEK293 cells were transiently transfected with plasmids encoding Payload C flanked by an ITR on either side, at least one Rep polypeptide, at least one Cap polypeptide, and at least one helper polypeptide, microtubule destabilizing agents were added 3 h hours post-transfection, and samples were taken at 3 days post-transfection. Different concentrations of microtubule destabilizing agents as shown in Table 1 were assessed. Fold changes in rAAV particle titer of about 1-fold to about 1.7-fold were obtained at 1× concentrations for all microtubule destabilizing agents (FIG. 6).

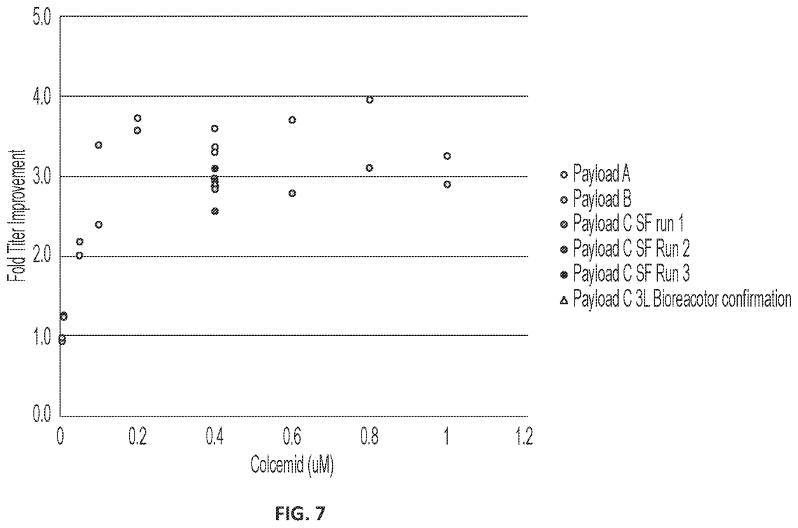

Improvement in titer of rAAV particles with different payloads after addition of colcemid was also determined. Suspension HEK293 cells were transiently transfected with plasmids encoding different payloads (A-C), flanked by an ITR on either side, at least one Rep polypeptide, at least one Cap polypeptide, and at least one helper polypeptide as well as microtubule destabilizing agents were added at 0.5 hours post-transfection. Dose response curves of about 0.005 μM to about 4 μM colcemid were generated for plasmids encoding Payload A and Payload B. A concentration of about 0.4 μM colcemid was used for the plasmid encoding Payload C. Fold changes in rAAV particle titer of about 3-fold to about 4-fold were obtained for all payloads (FIG. 7).

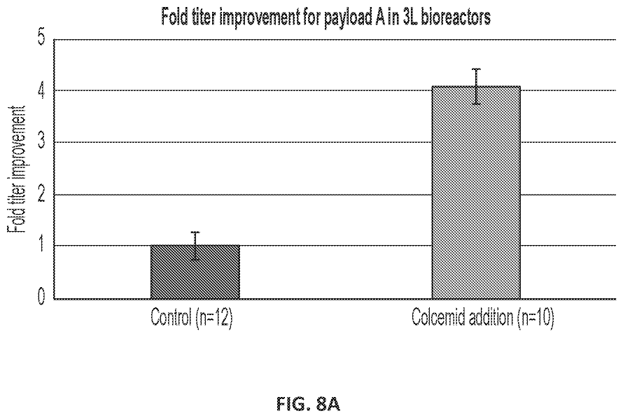

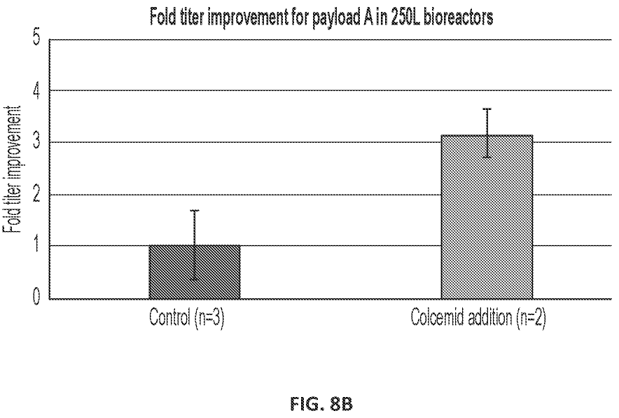

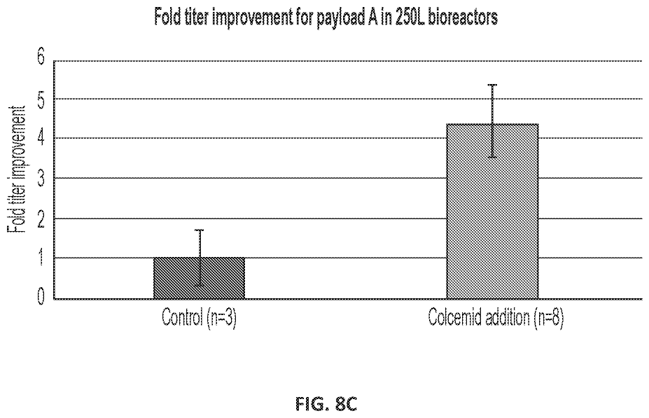

Scalability of microtubule destabilizing agents (colcemid and colchicine) for production of rAAV was also assessed in batch bioreactors and larger volume fed batch bioreactors for manufacturing scale. Suspension HEK293 cells were transiently transfected with plasmids encoding Payload A flanked by an ITR on either side, at least one Rep polypeptide, at least one Cap polypeptide, and at least one helper polypeptide, colcemid was added at 0.5 hours post-transfection, and cells were cultured in 3-liter batch bioreactors or 250-liter fed bioreactors for a period of time e.g., several days prior to sampling. Colcemid addition resulted in an increase in rAAV particle titer in both 3-liter batch bioreactors (FIG. 8A) and 250-liter fed bioreactors (FIG. 8B-8C) relative to controls.

These results provided herein demonstrate that the use of various microtubule destabilizing agents resulted in higher titers (e.g., genome titers) of rAAV particles relative to rAAV particles produced under the same conditions and in the same medium, but without a microtubule destabilizing agent. Thus, the present disclosure provides a rAAV production process using microtubule destabilizing agents that efficiently generates high titers of recombinant viral vector particles, which can be utilized at manufacturing scale.

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes described herein. The publications, websites and other reference materials referenced herein to describe the background of the invention and to provide additional detail regarding its practice are hereby incorporated by reference in their entireties.

We claim:

1. A method of producing a plurality of recombinant adeno-associated virus (rAAV) particles comprising:
(a) transfecting host cells with one or more vectors encoding:
(i) at least one payload flanked by an inverted terminal repeat (ITR) on either side of the at least one payload,
(ii) at least one Rep polypeptide,
(iii) at least one Cap polypeptide, and
(vii) at least one helper polypeptide, in medium;
(b) adding at least one microtubule destabilizing agent to the medium; and
(c) culturing the host cells under conditions suitable for production of the plurality of rAAV particles,
thereby producing the plurality of rAAV particles.

2. The method of claim 1, wherein the microtubule destabilizing agent comprises or is a G2/M inhibitor.

3. The method of claim 1 or 2, wherein the microtubule destabilizing agent comprises or is colcemid, colchicine, vinblastine, vincristine, or a combination, derivative, or salt thereof.

4. The method of any one of claims 1-3, wherein the microtubule destabilizing agent is added to the medium prior to transfection of the host cells with the one or more vectors.

5. The method of any one of claims 1-3, wherein the microtubule destabilizing agent is added to the medium substantially simultaneously with the one or more vectors.

6. The method of any one of claims 1-3, wherein the microtubule destabilizing agent is added to the medium after transfection of the host cells with the one or more vectors.

7. The method of claim 4, wherein the microtubule destabilizing agent is added to the medium at about 24 hours to about 1 minute prior to transfection.

8. The method of claim 6, wherein the microtubule destabilizing agent is added to the medium at about 24 hours to about 1 minute after transfection.

9. The method of any one of claims 1-8, wherein the plurality of rAAV particles are produced at a higher titer.

10. The method of claim 9, wherein the higher titer is relative to rAAV particles produced under the same conditions and in the same medium, but without a microtubule destabilizing agent.

11. The method of claim 9 or 10, wherein the higher titer comprises or is about a 1-fold higher titer to about a 6-fold higher titer.

12. The method of any one of claims 1-11, wherein the one or more vectors comprise:
(i) a first vector encoding at least one payload flanked by an ITR on either side of the at least one payload,
(ii) a second vector encoding at least one Rep polypeptide and at least one Cap polypeptide, and
(iii) a third vector encoding at least one helper polypeptide.

13. The method of any one of claims 1-11, wherein the one or more vectors comprise:
(i) a first vector encoding at least one Cap polypeptide and at least one payload flanked by an ITR on either side of the at least one payload; and
(ii) a second vector encoding at least one helper polypeptide and at least one Rep polypeptide.

14. The method of any one of claims 1-13, wherein the at least one helper polypeptide comprises one, two, three, or four of E1, E2A, E4orf6, or VA RNA polypeptides.

15. The method of any one of claims 1-14, wherein the host cells comprise or are adherent cells.

16. The method of any one of claims 1-14, wherein the host cells comprise or are suspension cells.

17. The method of any one of claims 1-15, wherein the host cells comprise or are mammalian cells.

18. The method of claim 17, wherein the mammalian cells comprise or are HEK293 cells, CHO-K cells, HeLa cells, or a variant thereof.

19. The method of any one of claims 1-18, wherein the host cells comprise or express an E1 polypeptide.

20. The method of any one of claims 1-19, wherein the one or more vectors are transfected into the host cells in the presence of polyethylenimine (PEI).

21. A reaction mixture comprising one or more vectors encoding:
(i) at least one payload flanked by an inverted terminal repeat (ITR) on either side of the at least one payload,
(ii) at least one Rep polypeptide,
(iii) at least one Cap polypeptide, and
(vii) at least one helper polypeptide; and
a microtubule destabilizing agent.

22. The reaction mixture of claim 21, wherein the one or more vectors comprises:
(i) a first vector encoding at least one payload flanked by an ITR on either side of the at least one payload,
(ii) a second vector encoding one at least one Rep polypeptide and at least one Cap polypeptide, and
(iii) a third vector encoding at least one helper polypeptide.

23. The reaction mixture of claim 21, wherein the one or more vectors comprises:
(i) a first vector encoding at least one Cap polypeptide and at least one payload flanked by an ITR on either side of the at least one payload; and
(ii) a second vector encoding at least one helper polypeptide and at least one Rep polypeptide.

24. A culture comprising a plurality of host cells and the reaction mixture of any of one of claims 21-23.

25. A bioreactor comprising the culture of claim 24.

26. The bioreactor of claim 25, wherein the bioreactor comprises or is a continuous flow bioreactor, a batch process bioreactor, a perfusion bioreactor, or a fed batch bioreactor.
